(19) United States
(12) Patent Application Publication
(10) Pub. No.: US 2021/0369624 A1
(43) Pub. Date: Dec. 2, 2021

(54) SOLID ORAL DOSAGE FORM HAVING EXCELLENT DISSOLUTION PROPERTIES

(71) Applicant: Sumitomo Dainippon Pharma Co., Ltd., Osaka-shi, Osaka (JP)

(72) Inventors: Yasuhiro Matsui, Suita-shi, Osaka (JP); Mikihiro Sugiura, Tokyo (JP); Masaru Yoshida, Suita-shi, Osaka (JP)

(21) Appl. No.: 17/059,970

(22) PCT Filed: May 31, 2019
(86) PCT No.: PCT/JP2019/021680

§ 371 Date: Nov. 30, 2020

(30) Foreign Application Priority Data
Jun. 1, 2018 (JP) 2018-105971

(51) Int. Cl. A61K 9/28; A61K 31/5377

(52) U.S. Cl. CPC A61K 9/2866; A61K 31/5377; A61K 9/2813; A61K 9/2826; A61K 9/2893; A61K 9/284

(57) ABSTRACT

The present disclosure relates to a solid oral dosage form comprising: (i) (S)-4-amino-5-chloro-N-[{4-[(1-hydroxyacetyl-4-piperidinyl)methyl]-2-morpholinyl}methyl]-2-methoxybenzamide, a pharmaceutically acceptable salt thereof, or a hydrate or solvate of the same; (ii) a disintegrating agent; and (iii) a water-soluble polymer binder. The present disclosure also relates to a medicinal composition, a therapeutic agent and/or a preventive agent, which comprise the medicine according to the present disclosure, for treating and/or preventing digestive diseases, digestive symptoms, psychoneurological diseases or urinary diseases, a preferable example thereof being a solid oral dosage form.

### TECHNICAL FIELD

The present disclosure relates to a solid oral formulation, which has (S)-4-amino-5-chloro-N-[{4-[(1-hydroxyacetyl-4-piperidinyl)methyl]-2-morpholinyl}methyl]-2-methoxybenzamide (present compound) or a pharmaceutically acceptable salt thereof, or a hydrate or solvate thereof (drug of the present disclosure) as an active ingredient and is capable of stably releasing the active ingredient even after storage at a desire concentration.

### BACKGROUND ART

An orally administered solid formulation disintegrates into particles of a size at which a drug is readily absorbed within the digestive tract, and then the drug dissolves into the digestive fluid from the particles and are absorbed from the digestive tract. Thus, it is desirable to design a formulation so that the active ingredient can be stably released at a desired concentration. The drug availability is generally determined by orally administering a solid formulation to a human or animal, then collecting blood at a certain time interval, and measuring the amount of drug in the serum. However, it is difficult to evaluate the drug availability in a human or animal during the designing stages of a solid formulation from the viewpoint of ethics. Thus, in vitro dissolution testing method is specified by the Japanese Pharmacopoeia. The Japanese Pharmacopoeia specifies the dissolution testing method for the purpose of securing a certain level of quality in a solid formulation as well as preventing significant biological nonequivalence.

Non Patent Literature 1 describes that the dissolution rate of active ingredient is delayed by the temperature or humidity depending on the type of disintegrant in a solid oral formulation. As a test on the quality of a drug, a stability test is generally conducted by a long-term test (25° C./60% RH) and accelerated test (40° C./75% RH). The phenomenon of delayed dissolution rate of an active ingredient is observed after storage in some cases from an accelerated test. In such a case, an active ingredient may not be able to be released stably at a desired concentration.

Patent Literature 1 discloses an orally administered formulation comprising pregelatinized starch, water soluble excipient, and water soluble macromolecular binding agent with lurasidone as the active ingredient, wherein the oral formulation exhibits the same dissolution behavior even if the active ingredient content is varied. However, said document does not have any disclosure or suggestion regarding the dissolution behavior after storage under heated and humidified conditions.

### CITATION LIST

### Patent Literature

[PTL 1] Japanese Laid-Open Publication No. 2011-126915

### Non Patent Literature

[NPL 1] Iyakuhin no Kaihatu [Development of drugs] Vol. 12 pages 185-197 (Hirokawa-Shoten)

### SUMMARY OF INVENTION

### Solution to Problem

The present disclosure provides a stable formulation exhibiting the same dissolution behavior under heated and humidified conditions, even if the active ingredient content is changed. More specifically, the present disclosure provides a solid oral formulation, which is stable against heating or humidifying, exhibits no decrease in the dissolution ratio after storage, exhibits the same dissolution property even when the active ingredient content is increased, and is capable of releasing the active ingredient at a desired concentration.

The inventors found that an oral formulation comprising an active ingredient, a disintegrant, and a water soluble macromolecular binding agent exhibits a rapid dissolution property, stability against heating and humidifying, and no decrease in the dissolution ratio. The inventors also found that the dissolution ratio does not decrease even if the active ingredient content is changed. The inventors also newly discovered an effect of reducing or preventing the delay of drug dissolution due to a cellulose-based disintegrant or a combination of a cellulose-based disintegrant and pregelatinized starch. Further, the inventors found a solid oral formulation that is stable and capable of releasing an active ingredient at a desired concentration.

Specifically, the present disclosure provides the following items.

### [Item 1]

A composition for reducing or preventing the delay of drug dissolution, comprising a disintegrant.

### [Item 2]

The composition of item 1, wherein the delay of drug dissolution is reduced or prevented after storage.

### [Item 3]

The composition of item 1 or 2, wherein the delay of drug dissolution is reduced or prevented after an accelerated test (40° C./75% RH).

### [Item 4]

The composition of any one of items 1 to 3, wherein the drug is (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof.

### [Item 5]

The composition of any one of items 1 to 4, wherein the disintegrant comprises a cellulose-based disintegrant.

### [Item 6]

The composition of item 5, wherein the cellulose-based disintegrant is one selected from the group consisting of croscarmellose sodium, crystalline cellulose, and low substituted hydroxypropyl cellulose or a mixture of two or more thereof.

### [Item 7]

The composition of any one of items 1 to 6, wherein the disintegrant comprises croscarmellose sodium or a combination of low substituted hydroxypropyl cellulose and pregelatinized starch.

### [Item 8]

The composition of any one of items 1 to 7, wherein the disintegrant comprises croscarmellose sodium.

### [Item 9]

The composition of any one of items 1 to 8, wherein the disintegrant comprises pregelatinized starch.

### [Item 10]

The composition of any one of items 1 to 9, wherein the disintegrant comprises a combination of croscarmellose sodium and pregelatinized starch.

### [Item 11]

The composition of any one of items 1 to 10, wherein the disintegrant comprises a combination of low substituted hydroxypropyl cellulose and pregelatinized starch.

### [Item 12]

The composition of any one of items 1 to 11, which is used further in combination with a water soluble macromolecular binding agent.

### [Item 13]

The composition of item 12, wherein the water soluble macromolecular binding agent is selected from the group consisting of polyvinylpyrrolidone, copolyvidone, hydroxypropyl cellulose, and polyvinyl alcohol.

### [Item 14]

The composition of item 12 or 13, wherein the water soluble macromolecular binding agent is selected from the group consisting of hydroxypropyl cellulose and polyvinyl alcohol.

### [Item 15]

The composition of any one of items 7 to 14, wherein a portion of the pregelatinized starch that is soluble in cold water is 40% by weight or less.

### [Item 16]

The composition of any one of items 7 to 14, wherein a portion of the pregelatinized starch that is soluble in water is 40% by weight or less.

### [Item 17]

The composition of any one of items 1 to 16, wherein content of the disintegrant is 1 to 50% by weight with respect to 100% by weight of the composition.

### [Item 18]

The composition of any one of items 1 to 17, wherein content of the disintegrant other than the pregelatinized starch is 1 to 10% by weight with respect to 100% by weight of the composition.

### [Item 19]

The composition of any one of items 1 to 18, wherein content of the disintegrant other than the pregelatinized starch is 1 to 5% by weight with respect to 100% by weight of the composition.

### [Item 20]

The composition of any one of items 7 to 19, wherein content of the pregelatinized starch is 10 to 50% by weight with respect to 100% by weight of the composition.

### [Item 21]

The composition of any one of items 7 to 20, wherein content of the pregelatinized starch is 15 to 30% by weight with respect to 100% by weight of the composition.

### [Item 22]

The composition of any one of items 6 to 21, wherein content of the croscarmellose sodium is 1 to 10% by weight with respect to 100% by weight of the composition.

### [Item 23]

The composition of any one of items 4 to 22, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof is 1 to 30% by weight with respect to 100% by weight of the composition.

### [Item 24]

The composition of any one of items 4 to 23, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof is 5 to 20% by weight with respect to 100% by weight of the composition.

### [Item 25]

The composition of any one of items 4 to 24, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof is 5 to 15% by weight with respect to 100% by weight of the composition.

### [Item 26]

The composition of any one of items 1 to 25, further comprising an excipient.

### [Item 27]

The composition of item 26, wherein the excipient is a water soluble excipient.

### [Item 28]

The composition of item 27, wherein the water soluble excipient is mannitol.

### [Item 29]

The composition of any one of items 1 to 28, further comprising a lubricant.

### [Item 30]

The composition of item 29, wherein the lubricant is sodium stearyl fumarate.

### [Item 31]

The composition of any one of items 1 to 30, wherein the composition is prepared by granulating a mixed powder comprising the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof, the pregelatinized starch, the disintegrant, and the water soluble excipient by using a solution with the water soluble macromolecular binding agent dissolved therein.

### [Item 32]

The composition of any one of items 1 to 31, wherein a film coating is applied with a coating agent.

### [Item 33]

The composition of item 32, wherein the coating agent is selected from the group consisting of hypromellose, polyvinylpyrrolidone, and hydroxypropyl cellulose.

### [Item 34]

The composition of item 32 or 33, wherein the coating agent further comprises a plasticizer.

### [Item 35]

The composition of item 34, wherein the plasticizer is selected from the group consisting of polyethylene glycol, propylene glycol, triacetin, triethyl citrate, glycerin, glycerin fatty acid ester, and polyethylene glycol.

### [Item 36]

The composition of any one of items 32 to 35, wherein the coating agent further comprises a coloring agent.

### [Item 37]

The composition of item 36, wherein the coloring agent is titanium oxide and/or yellow iron sesquioxide.

### [Item 38]

The composition of any one of items 1 to 37 for treating and/or preventing a digestive system disease, a digestive system symptom, a neuropsychiatric disease, or a urinary system disease.

### [Item 39]

The composition of item 38, wherein the digestive system disease is an irritable bowel syndrome (IBS) with constipation or chronic constipation.

### [Item 40]

A solid oral formulation comprising:

(i) (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof;
(ii) a disintegrant; and
(iii) a water-soluble macromolecular binding agent.

### [Item 41]

The solid oral formulation of item 40, wherein the disintegrant comprises a cellulose-based disintegrant.

### [Item 42]

The solid oral formulation of item 41, wherein the cellulose-based disintegrant is one selected from the group consisting of croscarmellose sodium, crystalline cellulose, and low substituted hydroxypropyl cellulose or a mixture of two or more thereof.

### [Item 43]

The solid oral formulation of any one of items 40 to 42, wherein the disintegrant comprises croscarmellose sodium or a combination of low substituted hydroxypropyl cellulose and pregelatinized starch.

### [Item 44]

The solid oral formulation of any one of items 40 to 43, wherein the disintegrant comprises croscarmellose sodium.

### [Item 45]

The solid oral formulation of any one of items 40 to 44, wherein the disintegrant comprises pregelatinized starch.

### [Item 46]

The solid oral formulation of any one of items 40 to 45, wherein the disintegrant comprises a combination of croscarmellose sodium and pregelatinized starch.

### [Item 47]

The solid oral formulation of any one of items 40 to 46, wherein the disintegrant comprises a combination of low substituted hydroxypropyl cellulose and pregelatinized starch.

### [Item 48]

The solid oral formulation of any one of items 40 to 47, wherein the water soluble macromolecular binding agent is selected from the group consisting of polyvinylpyrrolidone, copolyvidone, hydroxypropyl cellulose, and polyvinyl alcohol.

### [Item 49]

The solid oral formulation of any one of items 40 to 48, wherein the water soluble macromolecular binding agent is selected from the group consisting of hydroxypropyl cellulose and polyvinyl alcohol.

### [Item 50]

The solid oral formulation of any one of items 43 to 49, wherein a portion of the pregelatinized starch that is soluble in cold water is 40% by weight or less.

### [Item 51]

The solid oral formulation of any one of items 43 to 49, wherein a portion of the pregelatinized starch that is soluble in water is 40% by weight or less.

### [Item 52]

The solid oral formulation of any one of items 40 to 51, wherein content of the disintegrant is 1 to 50% by weight with respect to 100% by weight of the formulation.

### [Item 53]

The solid oral formulation of any one of items 40 to 52, wherein content of the disintegrant other than the pregelatinized starch is 1 to 10% by weight with respect to 100% by weight of the formulation.

### [Item 54]

The solid oral formulation of any one of items 40 to 53, wherein content of the disintegrant other than the pregelatinized starch is 1 to 5% by weight with respect to 100% by weight of the formulation.

### [Item 55]

The solid oral formulation of any one of items 43 to 54, wherein content of the pregelatinized starch is 10 to 50% by weight with respect to 100% by weight of the formulation.

### [Item 56]

The solid oral formulation of any one of items 43 to 55, wherein content of the pregelatinized starch is 15 to 30% by weight with respect to 100% by weight of the formulation.

### [Item 57]

The solid oral formulation of any one of items 42 to 56, wherein content of the croscarmellose sodium is 1 to 10% by weight with respect to 100% by weight of the formulation.

### [Item 58]

The solid oral formulation of any one of items 40 to 57, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof is 1 to 30% by weight with respect to 100% by weight of the formulation.

### [Item 59]

The solid oral formulation of any one of items 40 to 58, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof is 5 to 20% by weight with respect to 100% by weight of the formulation.

### [Item 60]

The solid oral formulation of any one of items 40 to 59, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof is 5 to 15% by weight with respect to 100% by weight of the formulation.

### [Item 61]

The solid oral formulation of any one of items 40 to 60, further comprising an excipient.

### [Item 62]

The solid oral formulation of item 61, wherein the excipient is a water soluble excipient.

### [Item 63]

The solid oral formulation of item 62, wherein the water soluble excipient is mannitol.

### [Item 64]

The solid oral formulation of any one of items 40 to 63, further comprising a lubricant.

### [Item 65]

The solid oral formulation of item 64, wherein the lubricant is sodium stearyl fumarate.

### [Item 66]

The solid oral formulation of any one of items 40 to 65, wherein the solid oral formulation is prepared by granulating a mixed powder comprising the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof, the disintegrant, and the water soluble excipient by using a solution with the water soluble macromolecular binding agent dissolved therein.

### [Item 67]

The solid oral formulation of any one of items 40 to 66, wherein a film coating is applied with a coating agent.

### [Item 68]

The solid oral formulation of item 67, wherein the coating agent is selected from the group consisting of hypromellose, polyvinylpyrrolidone, and hydroxypropyl cellulose.

### [Item 69]

The solid oral formulation of item 67 or 68, wherein the coating agent further comprises a plasticizer.

### [Item 70]

The solid oral formulation of item 69, wherein the plasticizer is selected from the group consisting of polyethylene glycol, propylene glycol, triacetin, triethyl citrate, glycerin, glycerin fatty acid ester, and polyethylene glycol.

### [Item 71]

The solid oral formulation of any one of items 67 to 70, wherein the coating agent further comprises a coloring agent.

### [Item 72]

The solid oral formulation of item 71, wherein the coloring agent is titanium oxide and/or yellow iron sesquioxide.

### [Item 73]

The solid oral formulation of any one of items 40 to 72 for treating and/or preventing a digestive system disease, a digestive system symptom, a neuropsychiatric disease, or a urinary system disease.

### [Item 74]

The solid oral formulation of item 73, wherein the digestive system disease is an irritable bowel syndrome (IBS) with constipation or chronic constipation.

### [Item 75]

A method of treating and/or preventing a digestive system disease, a digestive system symptom, a neuropsychiatric disease, or a urinary system disease, comprising administering a therapeutically and/or prophylactically effective amount of the solid oral formulation of any one of items 40 to 74 to a patient in need thereof.

### [Item 76]

The method of item 75, wherein the digestive system disease is an irritable bowel syndrome (IBS) with constipation or chronic constipation.

### [Item 77]

A composition for reducing or preventing the delay of drug dissolution, comprising a disintegrant.

### [Item 78]

The composition of item 77, wherein the delay of drug dissolution is delay of drug dissolution upon oral administration of a formulation comprising the drug.

### [Item 79]

The composition of item 77 or 78, wherein the delay of drug dissolution is reduced or prevented after storage.

### [Item 80]

The composition any one of items 77 to 79, wherein the delay of drug dissolution is reduced or prevented after an accelerated test (40° C./75% RH).

### [Item 81]

The composition of any one of items 77 to 80, wherein the drug is (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof.

### [Item 82]

The composition of any one of items 77 to 81, wherein the disintegrant comprises a cellulose-based disintegrant.

### [Item 83]

The composition of item 82, wherein the cellulose-based disintegrant is one selected from the group consisting of croscarmellose sodium, crystalline cellulose, and low substituted hydroxypropyl cellulose or a mixture of two or more thereof.

### [Item 84]

The composition of any one of items 77 to 83, wherein the disintegrant comprises croscarmellose sodium or a combination of low substituted hydroxypropyl cellulose and pregelatinized starch.

### [Item 85]

The composition of any one of items 77 to 84, wherein the disintegrant comprises croscarmellose sodium.

### [Item 86]

The composition of any one of items 77 to 85, wherein the disintegrant comprises pregelatinized starch.

### [Item 87]

The composition of any one of items 77 to 86, wherein the disintegrant comprises a combination of croscarmellose sodium and pregelatinized starch.

### [Item 88]

The composition of any one of items 77 to 87, wherein the disintegrant comprises a combination of low substituted hydroxypropyl cellulose and pregelatinized starch.

### [Item 89]

The composition of any one of items 77 to 88, which is used further in combination with a water soluble macromolecular binding agent.

### [Item 90]

The composition of item 89, wherein the water soluble macromolecular binding agent is selected from the group consisting of polyvinylpyrrolidone, copolyvidone, hydroxypropyl cellulose, and polyvinyl alcohol.

### [Item 91]

The composition of item 89 or 90, wherein the water soluble macromolecular binding agent is selected from the group consisting of hydroxypropyl cellulose and polyvinyl alcohol.

### [Item 92]

The composition of any one of items 84 to 91, wherein a portion of the pregelatinized starch that is soluble in cold water is 40% by weight or less.

### [Item 93]

The composition of any one of items 84 to 91, wherein a portion of the pregelatinized starch that is soluble in water is 40% by weight or less.

### [Item 94]

The composition of any one of items 77 to 93, wherein content of the disintegrant in a formulation comprising the disintegrant is 1 to 50% by weight with respect to 100% by weight of the formulation.

### [Item 95]

The composition of any one of items 77 to 94, wherein content of the disintegrant other than the pregelatinized starch in a formulation comprising the disintegrant is 1 to 10% by weight with respect to 100% by weight of the formulation.

### [Item 96]

The composition of any one of items 77 to 95, wherein content of the disintegrant other than the pregelatinized starch in a formulation comprising the disintegrant is 1 to 5% by weight with respect to 100% by weight of the formulation.

### [Item 97]

The composition of any one of items 84 to 96, wherein content of the pregelatinized starch in a formulation comprising the disintegrant is 10 to 50% by weight with respect to 100% by weight of the formulation.

### [Item 98]

The composition of any one of items 84 to 97, wherein content of the pregelatinized starch in a formulation comprising the disintegrant is 15 to 30% by weight with respect to 100% by weight of the formulation.

### [Item 99]

The composition of any one of items 83 to 98, wherein content of the croscarmellose sodium in a formulation comprising the disintegrant is 1 to 10% by weight with respect to 100% by weight of the formulation.

### [Item 100]

The composition of any one of items 81 to 99, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof in a formulation comprising the disintegrant is 1 to 30% by weight with respect to 100% by weight of the formulation.

### [Item 101]

The composition of any one of items 81 to 100, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof in a formulation comprising the disintegrant is 5 to 20% by weight with respect to 100% by weight of the formulation.

### [Item 102]

The composition of any one of items 81 to 101, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof in a formulation comprising the disintegrant is 5 to 15% by weight with respect to 100% by weight of the formulation.

### [Item 103]

The composition of any one of items 77 to 102, wherein a formulation comprising the disintegrant further comprises an excipient.

### [Item 104]

The composition of item 103, wherein the excipient is a water soluble excipient.

### [Item 105]

The composition of item 104, wherein the water soluble excipient is mannitol.

### [Item 106]

The composition of any one of items 77 to 105, wherein a formulation comprising the disintegrant further comprises a lubricant.

### [Item 107]

The composition of item 106, wherein the lubricant is sodium stearyl fumarate.

### [Item 108]

The composition of any one of items 77 to 107, wherein a formulation comprising the disintegrant is prepared by granulating a mixed powder comprising the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof, the pregelatinized starch, the disintegrant, and the water soluble excipient by using a solution with the water soluble macromolecular binding agent dissolved therein.

### [Item 109]

The composition of any one of items 77 to 108, wherein a film coating is applied to a formulation comprising the disintegrant with a coating agent.

### [Item 110]

The composition of item 109, wherein the coating agent is selected from the group consisting of hypromellose, polyvinylpyrrolidone, and hydroxypropyl cellulose.

### [Item 111]

The composition of item 109 or 110, wherein the coating agent further comprises a plasticizer.

### [Item 112]

The composition of item 111, wherein the plasticizer is selected from the group consisting of polyethylene glycol, propylene glycol, triacetin, triethyl citrate, glycerin, glycerin fatty acid ester, and polyethylene glycol.

### [Item 113]

The composition of any one of items 109 to 112, wherein the coating agent further comprises a coloring agent.

### [Item 114]

The composition of item 113, wherein the coloring agent is titanium oxide and/or yellow iron sesquioxide.

### [Item 115]

The composition of any one of items 77 to 114 for treating and/or preventing a digestive system disease, a digestive system symptom, a neuropsychiatric disease, or a urinary system disease.

### [Item 116]

The composition of item 115, wherein the digestive system disease is an irritable bowel syndrome (IBS) with constipation or chronic constipation.

### [Item 117]

Use of a disintegrant for reducing or preventing the delay of drug dissolution.

### [Item 118]

The use of item 117, wherein the delay of drug dissolution is delay of drug dissolution upon oral administration of a formulation comprising the drug.

### [Item 119]

The use of item 117 or 118, for reducing or preventing delay of drug dissolution after storage.

### [Item 120]

The use of any one of items 117 to 119, wherein the delay of drug dissolution is reduced or prevented after an accelerated test (40° C./75% RH).

### [Item 121]

The use of any one of items 117 to 120, wherein the drug is (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof.

### [Item 122]

The use of any one of items 117 to 121, wherein the disintegrant comprises a cellulose-based disintegrant.

### [Item 123]

The use of item 122, wherein the cellulose-based disintegrant is one selected from the group consisting of croscarmellose sodium, crystalline cellulose, and low substituted hydroxypropyl cellulose or a mixture of two or more thereof.

### [Item 124]

The use of any one of items 117 to 123, wherein the disintegrant comprises croscarmellose sodium or a combination of low substituted hydroxypropyl cellulose and pregelatinized starch.

### [Item 125]

The use of any one of items 117 to 124, wherein the disintegrant comprises croscarmellose sodium.

### [Item 126]

The use of any one of items 117 to 125, wherein the disintegrant comprises pregelatinized starch.

### [Item 127]

The use of any one of items 117 to 126, wherein the disintegrant comprises a combination of croscarmellose sodium and pregelatinized starch.

### [Item 128]

The use of any one of items 117 to 127, wherein the disintegrant comprises a combination of low substituted hydroxypropyl cellulose and pregelatinized starch.

### [Item 129]

The use of any one of items 117 to 128, which is used further in combination with a water soluble macromolecular binding agent.

### [Item 130]

The use of item 129, wherein the water soluble macromolecular binding agent is selected from the group consisting of polyvinylpyrrolidone, copolyvidone, hydroxypropyl cellulose, and polyvinyl alcohol.

### [Item 131]

The use of item 129 or 130, wherein the water soluble macromolecular binding agent is selected from the group consisting of hydroxypropyl cellulose and polyvinyl alcohol.

### [Item 132]

The use of any one of items 124 to 131, wherein a portion of the pregelatinized starch that is soluble in cold water is 40% by weight or less.

### [Item 133]

The use of any one of items 124 to 131, wherein a portion of the pregelatinized starch that is soluble in water is 40% by weight or less.

### [Item 134]

The use of any one of items 117 to 133, wherein content of the disintegrant in a formulation comprising the disintegrant is 1 to 50% by weight with respect to 100% by weight of the formulation.

### [Item 135]

The use of any one of items 117 to 134, wherein content of the disintegrant other than the pregelatinized starch in a formulation comprising the disintegrant is 1 to 10% by weight with respect to 100% by weight of the formulation.

### [Item 136]

The use of any one of items 117 to 135, wherein content of the disintegrant other than the pregelatinized starch in a formulation comprising the disintegrant is 1 to 5% by weight with respect to 100% by weight of the formulation.

### [Item 137]

The use of any one of items 124 to 136, wherein content of the pregelatinized starch in a formulation comprising the disintegrant is 10 to 50% by weight with respect to 100% by weight of the formulation.

### [Item 138]

The use of any one of items 124 to 137, wherein content of the pregelatinized starch in a formulation comprising the disintegrant is 15 to 30% by weight with respect to 100% by weight of the formulation.

### [Item 139]

The use of any one of items 123 to 138, wherein content of the croscarmellose sodium in a formulation comprising the disintegrant is 1 to 10% by weight with respect to 100% by weight of the formulation.

### [Item 140]

The use of any one of items 121 to 139, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof in a formulation comprising the disintegrant is 1 to 30% by weight with respect to 100% by weight of the formulation.

### [Item 141]

The use of any one of items 121 to 140, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof in a formulation comprising the disintegrant is 5 to 20% by weight with respect to 100% by weight of the formulation.

### [Item 142]

The use of any one of items 121 to 141, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof in a formulation comprising the disintegrant is 5 to 15% by weight with respect to 100% by weight of the formulation.

### [Item 143]

The use of any one of items 117 to 142, wherein a formulation comprising the disintegrant further comprises an excipient.

### [Item 144]

The use of item 143, wherein the excipient is a water soluble excipient.

### [Item 145]

The use of item 144, wherein the water soluble excipient is mannitol.

### [Item 146]

The use of any one of items 117 to 145, wherein a formulation comprising the disintegrant further comprises a lubricant.

### [Item 147]

The use of item 146, wherein the lubricant is sodium stearyl fumarate.

### [Item 148]

The use of any one of items 117 to 147, wherein a formulation comprising the disintegrant is prepared by granulating a mixed powder comprising the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof, the pregelatinized starch, the disintegrant, and the water soluble excipient by using a solution with the water soluble macromolecular binding agent dissolved therein.

### [Item 149]

The use of any one of items 117 to 148, wherein a film coating is applied to a formulation comprising the disintegrant with a coating agent.

### [Item 150]

The use of item 149, wherein the coating agent is selected from the group consisting of hypromellose, polyvinylpyrrolidone, and hydroxypropyl cellulose.

### [Item 151]

The use of item 149 or 150, wherein the coating agent further comprises a plasticizer.

### [Item 152]

The use of item 151, wherein the plasticizer is selected from the group consisting of polyethylene glycol, propylene glycol, triacetin, triethyl citrate, glycerin, glycerin fatty acid ester, and polyethylene glycol.

### [Item 153]

The use of any one of items 149 to 152, wherein the coating agent further comprises a coloring agent.

### [Item 154]

The use of item 153, wherein the coloring agent is titanium oxide and/or yellow iron sesquioxide.

### [Item 155]

The use of any one of items 117 to 154 for treating and/or preventing a digestive system disease, a digestive system symptom, a neuropsychiatric disease, or a urinary system disease.

### [Item 156]

The use of item 155, wherein the digestive system disease is an irritable bowel syndrome (IBS) with constipation or chronic constipation.

### Advantageous Effects of Invention

The inventors found that the solid oral formulation comprising a drug, disintegrant, and a water soluble macromolecular binding agent of the present disclosure exhibits a fast dissolution property, has stability against heating and humidifying, and has no decrease in the dissolution ratio. The inventors further found that the solid oral formulation of the present disclosure does not decrease the dissolution ratio against heating and humidifying even if the active ingredient content is changed. The inventors also newly found an effect of reducing or preventing the delay of drug dissolution due to a cellulose-based disintegrant or a combination of a cellulose-based disintegrant and pregelatinized starch. The inventors found a solid oral formulation, which is stable and capable of releasing an active ingredient at a desired concentration.

### BRIEF DESCRIPTION OF DRAWINGS

FIG. 1 shows the dissolution profile of Example 1.

FIG. 2 shows the dissolution profile of Example 2.

FIG. 3 shows the dissolution profile of Comparative Example 1.

FIG. 4 shows the dissolution profile of Comparative Example 2.

FIG. 5 shows the dissolution profile of Comparative Example 3.

FIG. 6 shows the dissolution profile of Example 3.

FIG. 7 shows the dissolution profile of Example 4.

FIG. 8 shows the dissolution profile of Example 5.

FIG. 9 shows the dissolution profile of Example 6.

FIG. 10 shows the dissolution profile of Example 7.

FIG. 11 shows the dissolution profile of Example 8.

### DESCRIPTION OF EMBODIMENTS

The present disclosure is described hereinafter in more detail. Throughout the entire specification, a singular expression should be understood as encompassing the concept thereof in the plural form, unless specifically noted otherwise. Thus, singular articles (e.g., “a”, “an”, “the”, and the like in the case of English) should also be understood as encompassing the concept thereof in the plural form, unless specifically noted otherwise. Further, the terms used herein should be understood as being used in the meaning that is commonly used in the art, unless specifically noted otherwise. Therefore, unless defined otherwise, all terminologies and scientific technical terms that are used herein have the same meaning as the general understanding of those skilled in the art to which the present disclosure pertains. In case of a contradiction, the present specification (including the definitions) takes precedence.

The terms herein are described hereinafter with the preferred embodiments. It is understood that the embodiments provided hereinafter are provided for better understanding of the present disclosure, so that the scope of the present disclosure is not limited to the following descriptions. It is therefore apparent that those skilled in the art can refer to the descriptions herein to make appropriate modifications within the scope of the present disclosure. It is understood that the following embodiments of the invention can be used alone or as a combination thereof.

As used herein, “mean particle size” refers to the cumulative 50% point of particle size D50 in a volume based measurement of powder particles. Such a mean particle size is measured based on volume with a laser diffraction granularity distribution analyzer (e.g., Powrex Corp: Particle Viewer, Shimadzu Corporation: SALD-3000J, or Sympatec: HELOS & RODOS).

As used herein, “content” refers to the amount added or content (wt/wt) that assumes the total amount of the formulation to be 100% by weight, unless specifically noted otherwise.

[1] Solid Oral Formulation

The solid oral formulation of the present disclosure comprises (i) a drug, (ii) a disintegrant, and (iii) a water soluble macromolecular binding agent, and may optionally comprise (iv) an excipient, (v) a lubricant, and/or (vi) an additive.

### (i) Drug

As used herein, “drug” refers to the active ingredient of the present disclosure, i.e., (S)-4-amino-5-chloro-N-[{4-[(1-hydroxyacetyl-4-piperidinyl)methyl]-2-morpholinyl}methyl]-2-methoxybenzamide (present compound) or a pharmaceutically acceptable salt thereof, or a hydrate or solvate thereof. This means that a drug can be in a form of a hydrate or solvate.

(S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof is a serotonin 4 receptor agonist, which is effective as a therapeutic agent or prophylactic agent for a digestive system disease, a digestive system symptom, a neuropsychiatric disease, or a urinary system disease.

A pharmaceutically acceptable salt is preferably hydrochloride or bromate, and more preferably bromate.

The content of drug of the present disclosure is generally 0.1 to 96% by weight, preferably 0.5 to 70% by weight, more preferably 1 to 30% by weight, still more preferably 5 to 20% by weight, and most preferably 5 to 15% by weight, with respect to 100% by weight of formulation.

A drug is preferably in a fine powder form. The mean particle size of a drug is generally 0.1 to 100 μm, preferably 0.1 to 80 μm, more preferably 0.1 to 50 μm, still more preferably 0.5 to 30 μm, and most preferably 1 to 25 μm. The mean particle size of a drug only needs to be within the range described above as a raw material and can change during the course of manufacture or the like. In another preferred embodiment, 90% or more of the particles by volume is preferably 40 μm or less.

### (ii) Disintegrant

“Disintegrant” refers to an agent added for the purpose of inducing disintegration or dispersion of a solid formulation such as a tablet or granule into particles.

Examples of “disintegrant” include croscarmellose sodium, pregelatinized starch, corn starch, crystalline cellulose, low substituted hydroxypropyl cellulose, carmellose, carmellose calcium, carmellose sodium, sodium carboxymethyl starch, carboxymethyl ethyl cellulose, crospovidone, and the like.

Preferred examples of “disintegrant” include croscarmellose sodium, pregelatinized starch, corn starch, low substituted hydroxypropyl cellulose, carmellose, carmellose calcium, carmellose sodium, sodium carboxymethyl starch, and crospovidone.

More preferred examples of “disintegrant” include croscarmellose sodium, pregelatinized starch, corn starch, low substituted hydroxypropyl cellulose, carmellose calcium, carmellose sodium, croscarmellose sodium, sodium carboxymethyl starch, and crospovidone.

Still more preferred examples of “disintegrant” include croscarmellose sodium, pregelatinized starch, low substituted hydroxypropyl cellulose, sodium carboxymethyl starch, and crospovidone.

Preferred examples of “disintegrant” include “cellulose-based disintegrant”. Examples of “cellulose-based disintegrant” include croscarmellose sodium, crystalline cellulose, low substituted hydroxypropyl cellulose, carmellose, carmellose calcium, carmellose sodium, carboxymethyl ethyl cellulose, and the like.

Preferred examples of “cellulose-based disintegrant” include croscarmellose sodium, crystalline cellulose, low substituted hydroxypropyl cellulose, and carmellose.

More preferred examples of “cellulose-based disintegrant” include croscarmellose sodium, crystalline cellulose, and low substituted hydroxypropyl cellulose.

Still more preferred examples of “cellulose-based disintegrant” include croscarmellose sodium and low substituted hydroxypropyl cellulose.

The most preferred example of “cellulose-based disintegrant” include croscarmellose sodium.

Thus, the most preferred example of “disintegrant” include croscarmellose sodium.

Another preferred embodiment of “disintegrant” includes a combination of croscarmellose sodium and pregelatinized starch. Still another preferred embodiment of “disintegrant” includes a combination of low substituted hydroxypropyl cellulose and pregelatinized starch. Although not wishing to be bound by any theory, this is because delay of the drug dissolution rate is reduced or does not happen when croscarmellose sodium alone, a combination of croscarmellose sodium and pregelatinized starch, or a combination of low substituted hydroxypropyl cellulose and pregelatinized starch is used as a disintegrant.

One disintegrant described above can be used or two or more disintegrants described above can be used simultaneously. Specifically, a disintegrant can be one selected from those described above or a mixture of two or more thereof, or a combination of two or more thereof.

The disintegrant content is generally 0.1 to 80% by weight, preferably 0.5 to 70% by weight, more preferably 0.5 to 60% by weight, still more preferably 1 to 50% by weight, and most preferably 1 to 40% by weight, per 100% by weight of formulation.

In another preferred embodiment, the disintegrant content is generally 1 to 50% by weight, preferably 1 to 40% by weight, more preferably 5 to 35% by weight, still more preferably 10 to 30% by weight, and most preferably 15 to 25% by weight, per 100% by weight of formulation.

Regarding content of the disintegrant, when two or more disintegrants are used, the combined content of two or more disintegrants is in the range described above.

Content of a disintegrant other than pregelatinized starch (e.g., cellulose-based disintegrant such as croscarmellose sodium or low substituted hydroxypropyl cellulose) is generally 0.1 to 50% by weight, preferably 0.5 to 40% by weight, more preferably 1 to 20% by weight, still more preferably 1 to 10% by weight, and most preferably 1 to 5% by weight, per 100% by weight of formulation.

Regarding content of the disintegrant other than pregelatinized starch, when two or more disintegrants other than pregelatinized starch are used, the combined content of two or more disintegrants other than pregelatinized starch is in the range described above.

The mean particle size of a disintegrant is generally 0.1 to 500 μm, preferably 1 to 300 μm, more preferably 10 to 200 μm, still more preferably 10 to 100 μm, and most preferably 20 to 100 μm. The mean particle size of a disintegrant only needs to be within the range described above as a raw material and can change during the course of manufacture or the like.

The “croscarmellose sodium” described above is a sodium salt of crosslinked multivalent carboxymethyl ether of cellulose. Examples thereof include the “croscarmellose sodium” described in the Japanese Pharmacopoeia and the like. Specific examples of “croscarmellose sodium” include Ac-Di-Sol® (FMC Bio Polymer), Primellose® (DFE Pharma), KICCOLATE® (Asahi Kasei Corporation), and the like.

The “low substitution hydroxypropyl cellulose” described above is low substituted hydroxypropyl ether of cellulose. Examples thereof include the “low substituted hydroxypropyl cellulose” described in the Japanese Pharmacopoeia and the like. Specific examples of “low substituted hydroxypropyl cellulose” include L-HPC® LH-21 (Shin-Etsu Chemical Co., Ltd.) and the like.

The “pregelatinized starch” described above is prepared by pregelatinizing various starches such as corn starch, potato starch, wheat starch, rice starch, and tapioca starch.

Specific examples of “pregelatinized starch” include the “Pregelatinized starch” and “Partly pregelatinized starch” in the Japanese Pharmaceutical Excipients, “Pregelatinized starch” in USP/NF, “Starch, pregelatinized” in Ph. Eur., and the like. “pregelatinized starch” is preferably “partly pregelatinized starch”. Specific examples of commercially available pregelatinized starch or partly pregelatinized starch include PCS (product name, distributor: Asahi Kasei Corporation), SWELSTAR (product name, distributor: Asahi Kasei Corporation), Starch 1500 and Starch 1500G (product name, distributor: Colorcon), LYCATAB C (product name, distributor: Roquette), and the like.

The pregelatinized starch content is generally 0.1 to 96% by weight, preferably 1 to 70% by weight, more preferably 5 to 50% by weight, still more preferably 10 to 50% by weight, even more preferably 10 to 30%, still even more preferably 15 to 30% by weight, and most preferably 15 to 25% by weight, per 100% by weight of formulation.

The mean particle size of pregelatinized starch is generally 0.1 to 500 μm, preferably 1 to 300 μm, more preferably 10 to 200 μm, still more preferably 10 to 100 μm, and most preferably 20 to 100 μm. The mean particle size of pregelatinized starch only needs to be within the range described above as a raw material and can change during the course of manufacture or the like.

A portion of the pregelatinized starch that is soluble in cold water is generally 40% by weight or less, preferably 30% by weight or less, more preferably 20% by weight of less, still more preferably 15% by weight of less, and most preferably 10% by weight or less.

A “portion soluble in cold water (% by weight)” in the present disclosure can be measured as follows. 3 g of sample (on dry basis) is precisely weight out, and 297 mL of 25° C. purified water is added. The mixture is stirred at a high speed for 2 minutes or longer at 800 rpm. The resulting suspension is transferred to a round bottom centrifuge tube and centrifuged for 15 minutes at 2000 rpm. 30 mL of supernatant is poured into a weighing bottle and dried until the weight settles at a certain value at 105° C. The portion soluble in cold water (% by weight) is a value obtained from dividing the weight of dried substance in the weighing bottle increased 1000-fold by the initial dry weight of the sample.

In the present disclosure, “pregelatinized starch with a portion soluble in cold water of 40% by weight or less” refers to partly pregelatinized starch with a portion contained in the supernatant, i.e., a water dissolving portion, of 40% by weight or less when a portion soluble in cold water is measured in accordance with the measurement method described above.

A portion of pregelatinized starch that is soluble in water is, from using the iodine coloimetric method, generally 40% by weight or less, preferably 30% by weight or less, more preferably 20% by weight or less, still more preferably 15% by weight of less, and most preferably 10% by weight or less.

A “portion soluble in water (% by weight)” in the present disclosure can be measured as follows. 1 g of pregelatinized starch (“preferably “pregelatinized starch”) is dissolved in 50 g of distilled water and stirred for 10 minutes with a magnetic stirrer. The mixture is filtered using filter paper or centrifuged at 2000 rpm to obtain a solution containing water soluble starch. The amount of portion of each pregelatinized starch that is soluble in water is obtained from the residue by drying the 40 g of filtrate (dried at 105° C. and dried for an additional 1 hour). The solution containing water soluble starch is serially diluted with distilled water, and the portion soluble in water is changed in stages. 30 μL of iodine solution (including I₂ 0.2%, KI 2.0%) is added to each solution. The absorbance at 660 nm is measured by spectrometry, and calibration curves for the amount of water soluble starch and absorbance are created. 1 g of pregelatinized starch is then dissolved in 50 g of distilled water and stirred for 10 minutes with a magnetic stirrer. The mixture is filtered using filter paper or centrifuged at 2000 rpm to obtain a solution containing water soluble starch. 30 μL of iodine solution (including I₂ 0.2%, KI 2.0%) is added to the solution. The absorbance at 660 nm is measured by spectrometry, and the amount of water soluble starch is found from the calibration curves for the amount of water soluble starch and absorbance created in advance.

The degree of pregelatinization of pregelatinized starch is generally expressed as “portion soluble in cold water (% by weight)” herein, but the degree of pregelatinization can also be expressed as “portion soluble in water (% by weight)”. The degree of pregelatinization can also be expressed as a pregelatinization ratio. When the pregelatinization ratio is used herein, a pregelatinization ratio measured by the glucoamylase method is generally used herein.

A pregelatinization ratio of pregelatinized starch is generally 40 to 100%, preferably 40 to 99%, more preferably 45 to 85%, still more preferably 50 to 80%, and most preferably 55 to 80%.

The pregelatinization ratio of partly pregelatinized starch is generally 40 to 99%, preferably 40 to 90%, more preferably 45 to 80%, still more preferably 50 to 75%, and most preferably 55 to 70%.

(iii) Water Soluble Macromolecular Binding Agent

“Binding agent” refers to an agent that imparts a binding force to powder and is used to shape and maintain the powder in a form of a formulation. “Water soluble macromolecular binding agent” refers to the “binding agent” described above, which is a naturally occurring or synthetic macromolecular compound with water solubility.

Examples of “water soluble macromolecular binding agent” include methyl cellulose, hydroxypropyl cellulose, hydroxypropyl methylcellulose, hydroxyethyl cellulose, hydroxymethyl cellulose, carboxymethyl cellulose, polyvinylpyrrolidone, polyvinyl alcohol, copolyvidone, polyethylene glycol, polyvinyl alcohol/acrylic acid/methyl methacrylate copolymer, vinyl acetate/vinylpyrrolidone copolymer, polyvinyl alcohol/polyethylene glycol/graft copolymer, pregelatinized starches other than the (ii) Pregelatinized starch described above, dextrin, dextran, pullulan, alginic acid, gelatin, and pectin.

One or two or more water soluble macromolecular binding agents described above can be used simultaneously. Specifically, a water soluble macromolecular binding agent can be one selected from those described above or a mixture of two or more thereof, or a combination of two or more thereof.

More preferred examples of water soluble macromolecular binding agents include hydroxypropyl cellulose, hydroxyethyl cellulose, polyvinylpyrrolidone, copolyvidone, and polyvinyl alcohol.

More preferred examples of water soluble macromolecular binding agents include hydroxypropyl cellulose, polyvinylpyrrolidone, copolyvidone, and polyvinyl alcohol.

Still more preferred examples of water soluble macromolecular binding agents include hydroxypropyl cellulose, copolyvidone, and polyvinyl alcohol.

The most preferred examples of water soluble macromolecular binding agents include hydroxypropyl cellulose and polyvinyl alcohol. Although not wishing to be bound by any theory, this is because use of hydroxypropyl cellulose or polyvinyl alcohol as a water soluble macromolecular binding agent enables excellent preparation (i.e., granulating) of a drug containing granule during formulation and results in tablets with excellent usability and excellent disintegration property.

The water soluble macromolecular binding agent content is generally 0.1 to 50% by weight, preferably 0.5 to 40% by weight, more preferably 1 to 20% by weight, still more preferably 1 to 10% by weight, and most preferably 1 to 5% by weight, per 100% by weight of formulation.

When two or more water soluble macromolecular binding agents are used, the combined content of two or more water soluble macromolecular binding agents is in the range described above.

Granules can be prepared by dissolving the water soluble macromolecular binding agent of the present disclosure in a solvent such as water and spraying. Granules can be also be prepared by spraying a solvent such as water containing a water soluble macromolecular binding agent with other components. When preparing granules while spraying a solvent such as water containing other components, the mean particles size of the water soluble macromolecular binding agent is generally 0.1 to 500 μm, preferably 1 to 300 μm, and more preferably 1 to 100 μm. The mean particle size of a water soluble macromolecular binding agent only needs to be within the range described above as a raw material and can change during the course of manufacture or the like.

### (iv) Excipient

“Excipient” refers to an agent that is added to shape the form, increase the amount, or dilute and improve the usability of a drug when the “bulk” is not sufficient with just the base component upon preparation of a dosage form, where the excipient has a function of not just simply increasing the amount, but also improving the mixability of powder, improving granulating property upon preparation of particles in granules or the like, improving the fillability into a mortar, adherence, and fluidity upon tableting for tablets, improving the fillability into a capsule for capsule agents or the like.

Examples of “excipient” include water soluble excipients and non-water soluble excipients. An excipient is preferably a water soluble excipient.

While a water soluble excipient that is generally used in formulation can be used as the water soluble excipient, preferred examples thereof include sugars and sugar alcohol.

The type of sugars and sugar alcohol is not particularly limited. Examples thereof include D-mannitol, erythritol, xylitol, maltitol, sorbitol, lactose, white sugar, trehalose, and the like. Sugars or sugar alcohol are preferably D-mannitol, erythritol, lactose, or trehalose, more preferably D-mannitol or lactose. Sugars or sugar alcohol are most preferably D-mannitol. In this regard, one or two or more water soluble excipients described above can be used. Specifically, a water soluble excipient can be one selected from those described above or a mixture of two or more thereof, or a combination of two or more thereof.

The excipient content is generally 0.1 to 96% by weight, preferably 1 to 90% by weight, more preferably 10 to 80% by weight, still more preferably 30 to 80% by weight, and most preferably 50 to 80% by weight, per 100% by weight of formulation.

The mean particles size of an excipient is generally 0.1 to 500 μm, preferably 1 to 300 μm, and more preferably 10 to 200 μm. The mean particle size of an excipient only needs to be within the range described above as a raw material and can change during the course of manufacture or the like.

### (v) Lubricant

“Lubricant” refers to an agent added for the purpose of improving fluidity or fillability, prevent adhesion, or the like of powder when filling a capsule or tableting in the manufacturing process of a capsule or tablet.

Examples of lubricants include magnesium stearate, sodium stearyl fumarate, talc, polyethylene glycol, silica, hydrogenated vegetable oil, and the like. A lubricant is more preferably magnesium stearate or sodium stearyl fumarate, and still more preferably sodium stearyl fumarate.

The lubricant content is generally 0.1 to 50% by weight, preferably 0.5 to 40% by weight, more preferably 0.1 to 20% by weight, still more preferably 1 to 10% by weight, and most preferably 1 to 5% by weight, per 100% by weight of formulation.

The mean particle size of a lubricant is generally 0.1 to 100 μm, preferably 0.1 to 50 μm, more preferably 0.1 to 30 μm, still more preferably 0.5 to 25 μm, and most preferably 1 to 15 μm. The mean particle size of a lubricant only needs to be within the range described above as a raw material and can change during the course of manufacture or the like.

### (vi) Additive

“Additive” refers to an agent contained in a formulation other than the active ingredient, which is used for the purpose of increasing the efficacy of the active ingredient and formulation, facilitating formulation, stabilizing the quality, improving the usability, or the like.

To the formulation of the present disclosure, a nontoxic and inactive additive that is generally used in the field of pharmaceutical formulations can be added as needed to the extent that the properties of the formulation of the present disclosure are not affected. Examples of such additives include those that do not affect the therapeutic effect of the active ingredient of the present disclosure and are commonly used in oral formulations. Examples of additives include stabilizers, corrigents (taste), sweeteners, corrigents (odor), flavoring agents, antioxidants, antistatic agents, fluidizers, coloring agents, and the like.

An additive is preferably a stabilizer, corrigent (taste), sweetener, corrigent (odor), flavoring agent, fluidizer, or coloring agent.

An additive is more preferably a stabilizer, corrigent (taste), sweetener, corrigent (odor), flavoring agent, or fluidizer.

An additive is still more preferably a corrigent (taste) or sweetener.

An additive is most preferably a sweetener.

Examples of stabilizers include meglumine, L-arginine, gelatin, salts thereof, and the like.

Examples of sweeteners include sugars, sugar alcohol, natural sweeteners such as licorice extract, stevia extract, Siraitia grosvenorii extract, and thaumatin, and synthetic sweeteners such as aspartame, saccharin, sodium saccharin, dipotassium glycyrrhizate, sucralose, and acesulfame K. The sweetener used is preferably erythritol, sorbitol, maltitol, mannitol, xylitol, aspartame, saccharin, sodium saccharin, dipotassium glycyrrhizate, stevia extract, thaumatin, sucralose, or acesulfame K.

Examples of corrigents (taste), corrigents (odor), and flavoring agents include sweetener components such as white sugar, saccharin, and various fruit syrups, organic acids such as fumaric acid, citric acid, and tartaric acid, and fruit essence.

Examples of antioxidants include tocopherol, EDTA, and the like.

Examples of antistatic agents include magnesium aluminometasilicate and the like.

Examples of fluidizers include talc, light anhydrous silicic acid, magnesium aluminometasilicate, water containing silicon dioxide, and the like.

Examples of coloring agents include tar dyes, lake pigments, yellow iron sesquioxide, iron sesquioxide, titanium oxide, and the like. The coloring agent used is preferably yellow iron sesquioxide.

The additive content can be set to any value, but is 0.1 to 96% by weight, preferably 0.5 to 70% by weight, more preferably 1 to 50% by weight, still more preferably 1 to 25% by weight, and most preferably 5 to 15% by weight, per 100% by weight of formulation.

In another preferred embodiment, a formulation is free of an additive.

An additive is preferably powder. If an additive is powder, the mean particle size of the additive used as a raw material is generally 0.1 to 500 μm and preferably 1 to 300 μm.

The mean particle size of an additive only needs to be within the range described above as a raw material and can change during the course of manufacture or the like.

“Solid oral formulation” refers to an orally administered solid formulation with a certain shape. Examples of solid formulations include formulations in a dosage form such as a tablet, capsule, granule, fine powder, pill, powder, or the like.

The solid oral formulation of the present disclosure particularly refers to formulations in a tablet, capsule, granule, or fine powder form. A solid oral formulation is preferably a tablet or capsule and more preferably a tablet.

A film coating can be applied to the solid oral formulation (e.g., tablet) in the present disclosure with a coating agent for the purpose of facilitating dosing, preventing degradation of an active ingredient, or the like. In such a case, the solid oral formulation of the present disclosure is a film-coated tablet. A film coated tablet is generally a tablet formulated by applying film coating to an uncoated tablet with a suitable coating agent such as a macromolecular compound.

“Coating agent” refers to an agent which is used to coat the surface of a formulation for preventing contact with water, air, or light, masking odor or bitterness, imparting a pharmaceutical property such as sustained release or enteric release properties, or improving the outer appearance to improve product value.

Examples of coating agents include combinations of a substrate such as hypromellose, hydroxypropyl cellulose, polyvinylpyrrolidone, methacrylic acid copolymer L, methacrylic acid copolymer LD, methacrylic acid copolymer S, ammonioalkyl methacrylate copolymer RS (aminoalkyl methacrylate copolymer RS), or ethyl acrylate/methyl methacrylate copolymer and a plasticizer such as polyethylene glycol, propylene glycol, triacetin, triethyl citrate, glycerin, glycerin fatty acid ester, or polyethylene glycol. A coating agent is preferably hypromellose, polyvinylpyrrolidone, or hydroxypropyl cellulose, more preferably hypromellose or polyvinylpyrrolidone, and still more preferably hypromellose.

“Plasticizer” refers to an agent that can make a material soft and readily processable by adding to and mixing in with the material. Examples of plasticizers include polyethylene glycol, propylene glycol, triacetin, triethyl citrate, glycerin, glycerin fatty acid ester, polyethylene glycol, and the like.

An additive such as titanium oxide, talc, or coloring agent can also be added to a coating agent. Carnauba wax, talc, or the like can also be added as a glazing agent after film coating.

“Coloring agent” refers to an agent used for identification of a capsule, tablet, or the like, light proofing of contained drug, or added commercial value. Examples of coloring agents include tar dyes, lake pigments, yellow iron sesquioxide, iron sesquioxide, titanium oxide, and the like.

While preparation of the oral formulation of the present disclosure varies depending on the desired dosage form, a desired dosage form can be prepared in accordance with a conventional method.

### (1) Preparation of an Aqueous Solution of a Water Soluble Macromolecular Binding Agent:

A water soluble macromolecular binding agent is dissolved into purified water. The amount of water soluble macromolecular binding agent is selected, for example, from the range of 1 to 20% by weight, preferably 2 to 8% by weight with respect to the amount of the purified water.

### (2) Preparation of the Drug Containing Granules of the Present Disclosure:

Granules are prepared while spraying the water soluble macromolecular binding agent prepared in step (1) into a granulator filled with the drug, water soluble excipient, and disintegrant of the present disclosure. Granules can also be prepared while spraying a solvent such as water into a granulator filled with the drug, water soluble excipient, disintegrant, and water soluble macromolecular binding agent of the present disclosure.

Examples of granulators include, but are not limited to, granulators classified into fluid bed granulation, high-share granulation, roto fluid bed granulation, twin screw granulation granulators and the like.

### (3) Drying of Granules:

The granules described above are dried under reduced pressure or under normal pressure. Granules are dried so that the value of loss in weight from drying measured by an infrared moisture analyzer is, for example, 4% by weight or less, preferably 1 to 3% by weight or less.

### (4) Blending of Lubricant:

A lubricant is added to the granules dried in (3) and mixed. For example, a mixer classified as a diffusion mixer [tumble] is used for mixing. Specific examples thereof include, but are not limited to, tumbler blenders, V blenders, double cones, bin tumblers, and the like.

### (5) Tableting:

Tablets are prepared by tableting the mixture described above.

Examples of tableting apparatuses include tableting machines classified as a tablet press and the like. Tablet hardness is selected, for example, from the range of 30 to 200 N.

(6) Film Coating is Applied when Desired:

The tablets described above may be coated with a film as needed. Examples of coating apparatuses include apparatuses classified as a coating pan. Preferred examples thereof include apparatuses classified as a perforated coating system.

### (7) Drying:

Tablets obtained in the manner described above are dried. Tablets are dried under reduced pressure or normal pressure so that the value of loss in weight from drying measured by an infrared moisture analyzer is, for example, 4% by weight or less, preferably 1 to 3% by weight or less.

One embodiment provides a solid oral formulation comprising:

(i) (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof;
(ii) a disintegrant comprising a cellulose-based disintegrant; and
(iii) hydroxypropyl cellulose or polyvinyl alcohol as a water soluble macromolecular binding agent; and optionally containing (iv) an excipient, (v) a lubricant, and/or (vi) an additive.

A preferred embodiment provides a solid oral formulation comprising:

(i) (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof;
(ii) croscarmellose sodium or a combination of low substituted hydroxypropyl cellulose and pregelatinized starch as a disintegrant; and
(iii) hydroxypropyl cellulose or polyvinyl alcohol as a water soluble macromolecular binding agent; and optionally containing (iv) an excipient, (v) a lubricant, and/or (vi) an additive.

A more preferred embodiment provides a solid oral formulation comprising:

(i) (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof;
(ii) croscarmellose sodium as a disintegrant; and
(iii) hydroxypropyl cellulose or polyvinyl alcohol as a water soluble macromolecular binding agent; and optionally containing (iv) an excipient, (v) a lubricant, and/or (vi) an additive.

Another embodiment provides a solid oral formulation comprising:

(i) (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof;
(ii) a disintegrant comprising pregelatinized starch; and
(iii) hydroxypropyl cellulose or polyvinyl alcohol as a water soluble macromolecular binding agent; and optionally containing (iv) an excipient, (v) a lubricant, and/or (vi) an additive.

Another preferred embodiment provides a solid oral formulation comprising:

(i) (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof;
(ii) a combination of croscarmellose sodium and pregelatinized starch as a disintegrant; and
(iii) hydroxypropyl cellulose or polyvinyl alcohol as a water soluble macromolecular binding agent; and optionally containing (iv) an excipient, (v) a lubricant, and/or (vi) an additive.

A still another preferred embodiment provides a solid oral formulation comprising:

(i) (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof;
(ii) a combination of low substituted hydroxypropyl cellulose and pregelatinized starch as a disintegrant; and
(iii) hydroxypropyl cellulose or polyvinyl alcohol as a water soluble macromolecular binding agent; and optionally containing (iv) an excipient, (v) a lubricant, and/or (vi) an additive.

[2] Pharmaceutical Use of Solid Oral Formulation

The present disclosure relates to a pharmaceutical composition, therapeutic agent, and/or prophylactic agent, preferably a solid oral formulation, for treating and/or preventing a digestive system disease, a digestive system symptom, a neuropsychiatric disease, or a urinary system disease, comprising the drug of the present disclosure. Preferred examples of the digestive system disease include irritable bowel syndrome (IBS) with constipation and chronic constipation.

In the present disclosure, “prevention (prophylaxis)” is an act of administering the present compound, which is the active ingredient, to a healthy individual who has not developed a disease or does not have an unhealthy condition as of the administration. “Prophylactic agent” is administered to such a healthy individual. For example, a prophylactic agent is intended to prevent the development of a disease and is expected to be suitable for especially individuals who have had a symptom of a disease previously or individuals considered to have increased risk of suffering from the disease. “Therapy” is an act of administering the present compound, which is an active ingredient, to an individual (patient) diagnosed to have developed a disease by a physician. “Therapeutic agent” is administered to such a patient. For example, a therapeutic agent is intended to alleviate a disease or symptom, prevent exacerbation of a disease or symptom, or restore the condition to that prior to developing the disease. Even when the objective of administration is prevention of exacerbation of a disease or symptom, this is an act of therapy if the agent is administered to a patient.

In the present disclosure, specific examples of the diseases or symptoms described above include the diseases or symptoms of the following (i) to (v).

(i) digestive system diseases, such as irritable bowel syndrome, atonic constipation, habitual constipation, chronic constipation, constipation induced by agents such as morphine and antipsychotics, constipation accompanying Parkinson's disease, constipation accompanying multiple sclerosis, constipation accompanying diabetes, and constipation or defecation disorder due to a contrast agent (as an endoscopic examination or pretreatment for barium intestinal enema X-ray examination);
(ii) digestive system diseases such as functional dyspepsia, acute/chronic gastritis, reflux esophagitis, gastric ulcer, duodenal ulcer, gastric neurosis, postoperative paralytic ileus, senile ileus, non-diffuse gastroesophageal reflux disease, NSAID ulcer, diabetic gastroparesis, post-gastrectomy syndrome, and intestinal pseudo-obstruction;
(iii) digestive system symptoms such as anorexia, nausea, vomiting, bloating, epigastric discomfort, abdominal pain, heartburn, and eructation in the digestive system diseases described in (i) and (ii), scleroderma, diabetes, or esophagus/biliary tract disease;
(iv) neuropsychiatric diseases such as schizophrenia, Alzheimer's disease, depression, memory disorder, and anxiety; and
(v) urinary system diseases involving urinary disturbance due to prostatic hyperplasia, urinary tract obstruction, or the like.

In this manner, the compound of the present disclosure can be used in treating and preventing various diseases described above, especially various digestive system dysfunctions involving the treatment of various diseases described above or digestive system diseases. Specifically, the compound of the present disclosure exhibits excellent movement promoting action on the digestive tract (especially the lower digestive tract of the colon or rectum), so that the compound is useful as a digestive tract motion promoting drug or digestive tract function improving drug with a potent laxative action, especially as a therapeutic or prophylactic drug for a disease described in (i).

The dosage form of the present compound or a pharmaceutically acceptable salt thereof, or a hydrate or solvate thereof can be either oral administration or parenteral administration, but oral administration is preferred. The dosage varies depending on the dosing method, symptom or age of the patient or the like, but is generally in the range of 0.01 to 30 mg/kg/day, preferably 0.05 to 10 mg/kg/day, and more preferably 0.1 to 3 mg/kg/day. In another preferred embodiment, the dosage is generally in the range of 0.01 mg to 1000 mg/day, preferably 0.1 mg to 500 mg/day, more preferably 0.5 mg to 300 mg/day, still more preferably 1 mg to 200 mg/day, and most preferably 5 mg to 100 mg. The number of dosing per day is one or several, such as 1, 2, or 3 doses given each time.

Examples of orally administered formulations include tablets, capsules, granules, powder, syrup, fine powder, liquid agents, suspension, and the like. Examples of parenterally administered formulations include injection, intravenous drip agents, suppository (intrarectally administered agents), nasal agents, sublingual agents, transdermally absorbed agents [lotion, emulsion, ointment, cream, jelly, gel, patch (tape, transdermal patch formulation, poultice, and the like), externally applied powder, and the like], and the like.

A substance that is commonly used in the pharmaceutical field and does not react with the present compound or a pharmaceutically acceptable salt thereof, or a hydrate or solvate thereof is used as a carrier for a formulation. Specifically, a formulation comprising the present compound or a pharmaceutically acceptable salt thereof, or a hydrate or solvate thereof can comprise a carrier for a formulation such as an excipient, binding agent, lubricant, stabilizer, disintegrant, buffer, solubilizer, isotonizing agent, solubilizer, pH modifier, surfactant, emulsifier, suspending agent, dispersant, precipitation preventing agent, thickener, viscosity modifier, gelatinizer, analgesic, preservative, plasticizer, transdermal absorption promoting agent, antioxidant, humectant, antiseptic, or flavoring agent. Two or more carriers for a formulation can also be selected and used as appropriate.

Specific examples of carriers for a formulation include lactose, inositol, glucose, sucrose, fructose, mannitol (mannite), dextran, sorbitol, cyclodextrin, starch (potato starch, corn starch, amylopectin, etc.), partly pregelatinized starch, white sugar, magnesium aluminometasilicate, synthetic aluminum silicate, sodium alginate, crystalline cellulose, sodium carboxymethyl cellulose, hydroxypropyl starch, carboxymethyl cellulose calcium, ion exchange resin, methyl cellulose, gelatin, gum arabic, pullulan, hydroxypropyl cellulose, low substituted hydroxypropyl cellulose, hydroxypropyl methylcellulose, carboxymethyl cellulose, hydroxyethyl cellulose, polyvinylpyrrolidone, polyvinyl alcohol, gelatin, alginic acid, sodium alginate, light anhydrous silicic acid, magnesium stearate, calcium stearate, aluminum stearate, cetostearyl alcohol, wax, paraffin, talc, tragacanth, bentonite, Veegum, carboxyvinyl polymer, titanium oxide, fatty acid ester, sorbitan fatty acid ester, sodium lauryl sulfate, glycerin, fatty acid glycerin ester, refined lanolin, glycerinated gelatin, polysorbate, macrogol, squalane, silicone oil, vegetable oil (sesame oil, olive oil, soybean oil, cotton seed oil, castor oil, etc.), liquid paraffin, soft paraffin, white vaseline, yellow vaseline, paraffin, wool fat, wax (beeswax, carnauba wax, white beeswax, etc.), water, propylene glycol, polyethylene glycol, glycerol, lauryl alcohol, myristyl alcohol, oleyl alcohol, cetyl alcohol, ethanol, sodium chloride, sodium hydroxide, hydrochloric acid, citric acid, lauric acid, myristic acid, stearic acid, oleic acid, benzyl alcohol, glutamic acid, glycine, methyl paraoxybenzoate, propyl paraoxybenzoate, p-hydroxybenzoic acid ester, cholesterol ester, ethylene glycol monoalkyl ester, propylene glycol monoalkyl ester, glycerin monostearate, sorbitan fatty acid ester, isopropyl myristate, isopropyl palmitate, carboxypolymethylene, saccharin, strawberry flavor, peppermint flavor, cocoa butter, polyisobutylene, vinyl acetate copolymer, acrylic copolymer, triethyl citrate, acetyltriethyl citrate, diethyl phthalate, diethyl sebacate, dibutyl sebacate, acetylated monoglyceride, diethylene glycol, dodecylpyrrolidone, urea, ethyl laurate, Azone, kaolin, bentonite, zinc oxide, agarose, carrageenan, acacia gum, xanthan gum, potassium laurate, potassium palmitate, potassium myristate, sodium cetyl sulfate, castor oil sulfate (Turkey red oil), Span (sorbitan stearate, sorbitan monooleate, sorbitan sesquioleate, sorbitan trioleate, etc.), Tween (polysorbate 20, polysorbate 40, polysorbate 60, polysorbate 65, polysorbate 80, polysorbate 85, polyoxyethylene sorbitan fatty acid ester, etc.), polyoxyethylene hydrogenated castor oil (so-called HCO), polyoxyethylene lauryl ether, polyoxyethylene cetyl ether, polyoxyethylene oleyl ether, polyethylene glycol monolaurate, polyethylene glycol monostearate, poloxamer (so-called Pluronic), lecithin (including purified phospholipids isolated from lecithin such as phosphatidylcholine and phosphatidylserine), hydrogenated lecithin, and the like.

The present compound or a pharmaceutically acceptable salt thereof, or a hydrate or solvate thereof, when used for a pharmaceutical use described above, is generally administered in a form of a formulation prepared by mixing with a carrier for a formulation. The formulation is prepared in accordance with a normal method. For example, a pharmaceutical composition can comprise the present compound or a pharmaceutically acceptable salt thereof, or a hydrate or solvate thereof as an active ingredient at 0.01 to 99% by weight, preferably 0.05 to 80% by weight, more preferably 0.1 to 70% by weight, and still more preferably 0.1 to 50% by weight. These formulations can also comprise other therapeutically valuable components.

The present invention encompasses a combined therapy that administers the present compound or a pharmaceutically acceptable salt thereof, or a hydrate or solvate thereof, or a formulation comprising the same in combination with one or more other agents described below, sequentially or simultaneously, for the treatment of the disease described herein.

Specific examples of such agents, for a digestive system disease with constipation, include saline laxatives such as magnesium sulfate, magnesium oxide, and magnesium citrate, emollient laxatives such as dioctyl sodium sulfosuccinate and casanthranol, bulk-forming laxatives such as carmellose, large intestine stimulating laxatives such as bisacodyl, picosulfate, senna, and sennoside, small intestine stimulating laxatives such as castor oil, bowel cleansing agents such as Magcorol and Niflec, and the like.

Examples thereof include, for a digestive system disease such as functional dyspepsia, acute/chronic gastritis, reflux esophagitis, non-diffuse gastroesophageal reflux disease, gastroparesis diabeticorum, gastric ulcer, duodenal ulcer, NSAID ulcer, gastric neurosis, postoperative paralytic ileus, senile ileus, postgastrectomy syndrome, or intestinal pseudo-obstruction, proton pump inhibitors such as omeprazole, rabeprazole, and lansoprazole, antacids such as histamine H₂-receptor inhibitors such as cimetidine, ranitidine, and famotidine, intestinal tract function adjusting agents such as mosapride and domperidone, gastric mucosal protective agents, intestinal function controlling agents, and the like. Examples thereof include, for depression/anxiety, antidepressants/anxiolytics, e.g., selective serotonin reuptake inhibitors (SSRI) such as paroxetine and sertraline, serotonin-norepinephrine reuptake inhibitors (SNRI) such as venlafaxine and duloxetine, tricyclic antidepressants such as amitriptyline and imipramine, tetracyclic antidepressants such as mianserin and maprotiline. Examples thereof include, for memory disorders, cholinesterase inhibitors such as donepezil and rivastigmine and cognitive disorder improving drugs such as memantine. Examples thereof include, for urinary disturbance due to prostatic hyperplasia, urinary disturbance therapeutic drugs such as tamsulosin and terazosin.

[3] Composition for Reducing or Preventing Delay of Drug Dissolution and Use Thereof

The present disclosure relates to a composition for reducing or preventing the delay of drug dissolution, comprising a disintegrant. In a preferred example, the delay of drug dissolution is delay of drug dissolution upon oral administration of a formulation comprising the drug. The dissolution ratio of the drug of the present disclosure does not decrease after storage in a more preferred example, or after humidified or heated accelerated test (storage for 2 or 4 months in a 40° C./75% RH hygrostat incubator) or stress test (storage for 2 weeks in a 50° C./85′% RH hygrostat incubator) in a more preferred example.

In a preferred example, the present disclosure relates to a composition for reducing or preventing the delay of drug dissolution, comprising a disintegrant, wherein the drug is (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof. The composition is used further in combination with a water soluble macromolecular binding agent. In another embodiment, the composition is used in combination with both a disintegrant and a water soluble polymer binding agent. The aforementioned drug, disintegrant, and water soluble macromolecular binding agent indicate (i) drug, (ii) disintegrant, and (iii) water soluble macromolecular binding agent described above.

In one embodiment, the composition for reducing or preventing the delay of drug dissolution of the present disclosure comprises a cellulose-based disintegrant as (ii) a disintegrant. The composition further comprises (iii) a water soluble macromolecular binding agent and optionally comprises (iv) an excipient, (v) a lubricant, and/or (vi) an additive.

In one preferred embodiment, the composition for reducing or preventing the delay of drug dissolution of the present disclosure comprises croscarmellose sodium or a combination of low substituted hydroxypropyl cellulose and pregelatinized starch as (ii) a disintegrant. The composition further comprises (iii) a water soluble macromolecular binding agent and optionally comprises (iv) an excipient, (v) a lubricant, and/or (vi) an additive.

In a more preferred embodiment, the composition for reducing or preventing the delay of drug dissolution of the present disclosure comprises croscarmellose sodium as (ii) a disintegrant. The composition further comprises (iii) a water soluble macromolecular binding agent and optionally comprises (iv) an excipient, (v) a lubricant, and/or (vi) an additive.

In another embodiment, the composition for reducing or preventing the delay of drug dissolution of the present disclosure comprises pregelatinized starch as (ii) a disintegrant. The composition further comprises (iii) a water soluble macromolecular binding agent and optionally comprises (iv) an excipient, (v) a lubricant, and/or (vi) an additive.

In another preferred embodiment, the composition for reducing or preventing the delay of drug dissolution of the present disclosure comprises a combination of croscarmellose sodium and pregelatinized starch as (ii) a disintegrant. The composition further comprises (iii) a water soluble macromolecular binding agent and optionally comprises (iv) an excipient, (v) a lubricant, and/or (vi) an additive.

In still another preferred embodiment, the composition for reducing or preventing the delay of drug dissolution of the present disclosure comprises a combination of low substituted hydroxypropyl cellulose and pregelatinized starch as (ii) a disintegrant. The composition further comprises (iii) a water soluble macromolecular binding agent and optionally comprises (iv) an excipient, (v) a lubricant, and/or (vi) an additive.

The method of manufacturing such a composition for reducing or preventing the delay of drug dissolution can be any manufacturing method known in the art. An example thereof includes a manufacturing method for manufacturing granules by preparing a binding solution that can dissolve a water soluble macromolecular binding agent ((iii) described above) in a powder mixture comprising a raw material drug ((i) described above) and disintegrant ((ii) described above) and granulating while adding (e.g., spraying) the solution, and then tableting and manufacturing uncoated tablets and coating the tablets with a film.

A decrease in the dissolution ratio, i.e., delay of drug dissolution, can be evaluated to confirm the effect of reducing or preventing delay of drug dissolution by conducting a dissolution test on a composition for reducing or preventing the delay of drug dissolution immediately after the manufacture and after storage and comparing the dissolution behaviors thereof. The dissolution testing method can be any testing method that is known in the art. An example thereof includes a testing method in the Japanese Pharmacopoeia, Dissolution Test, Paddle Method (Apparatus 2). Furthermore, an accelerated test (storage for 2 or 4 months in a 40° C./75% RH hygrostat incubator) or stress test (storage for 2 weeks in a 50° C./85% RH hygrostat incubator) is conducted to evaluate the dissolute ratios immediately after manufacture and after storage. If the dissolution behavior after storage is the same excellent dissolution behavior as that immediately after manufacture, the composition is recognized as having an effect of reducing or preventing delay of drug dissolution.

As used herein, “storage” refers to leaving, stowing, or stocking a manufactured formulation in a suitable container. During storage, the container may be sealed or open, and shielded or not shielded from light. Examples of storage include storage at normal temperature, storage at room temperature, cold storage, refrigeration, freezing, and the like. Examples of temperatures during storage include, but are not limited to, standard temperature: 20° C., normal temperature: 15 to 25° C., room temperature: 1 to 30° C., lukewarm: 30 to 40° C., cold: 1 to 15° C., refrigerated: 2 to 6° C., freezing: about −20° C. to −18° C., and the like. The relative humidity (% RH) during storage can be, but not limited to, any of 0 to 100% RH and preferably 40 to 60% RH. Examples of storage period include, but are not limited to, several hours, days, weeks, months, years, and the like. Parameters for the storage described above can be appropriately selected depending on the properties of the formulation or storage conditions.

“Accelerated test” as used herein is a safety test for confirming whether the quality of manufactured formulations is retained using a storage condition that promotes a chemical change or physical change in the formulation. Results of an accelerated test can be used in evaluating a chemical effect from long term storage by a given storage method. At the same time, the results can also be used to evaluate the effect of short term deviation from the storage method that can occur during transport. An “accelerated test” can be conducted using a storage condition with a higher temperature/higher relative humidity (e.g., 40° C./75% RH) relative to a storage condition with a temperature/relative humidity that is used in normal long term tests (e.g., 25° C./60% RH). A “stress test”, as used herein, can be conducted using a higher temperature/higher relative humidity (e.g., 50° C./85% RH) relative to the storage condition for the “accelerated test” described above. Examples of storage periods of an “accelerated test” or “stress test” include, but are not limited to, the storage periods for the “storage” described above (e.g., 2 months and 2 weeks, respectively). The accelerated test or stress test herein is conducted by setting hygrostat incubator conditions to a higher temperature/higher relative humidity (e.g., 40° C./75% RH and 50° C./85% RH, respectively), placing a prepared solid formulation in a suitable container (e.g., HDPE bottle (material: high-density polyethylene (HDPE), volume: 30 ml, H: 61 mm, W: 35 mm, L: 30 mm)), and storing the container for a certain storage period (e.g., 2 months and 2 weeks, respectively) in a hygrostat incubator in an open state.

### EXAMPLES

The Examples of the present disclosure are provided hereinafter. The Examples do not limit the present disclosure in any manner, and are provided solely to describe the present disclosure. The present disclosure can also be modified to the extent that the invention remains within the scope of the present disclosure. The compound names denoted in the following Examples, Reference Examples, and Test Examples are not necessarily in accordance with the IUPAC nomenclature.

In the Examples, Reference Examples, and Test Example, % in a solvent indicates (W/W %) and % in particles indicates % by weight, unless specifically noted otherwise.

The following additives were used in the Examples, Reference Examples, and Test Example, unless specifically noted otherwise.

Hydroxypropyl cellulose: HPC-L (Nippon Soda Co., Ltd.) Croscarmellose sodium: Ac-Di-Sol® (FMC BioPolymer)
Mannitol: Mannite P (Mitsubishi Shoji Foodtech Co., Ltd.) Partly pregelatinized starch: PCS® (product name, distributor: Asahi Kasei Corporation)
Low substituted hydroxypropyl cellulose: L-HPC® LH-21 (Shin-Etsu Chemical Co., Ltd.)
Sodium stearyl fumarate: PRUV® (Rettenmaier Japan Co., Ltd.)
Polyvinyl alcohol: Gohsenol® EG-05P (The Nippon Synthetic Chemical Industry Co., Ltd.)
Hypromellose: TC-5® R (Shin-Etsu Chemical Co., Ltd.) Titanium oxide: titanium oxide (Watanabe Chemical Co., Ltd.)
Propylene glycol: Japanese Pharmacopoeia propylene glycol (ADEKA Corporation)
Yellow iron sesquioxide: yellow iron sesquioxide (San-Ei Gen F.F.I., Inc.)

### Example 1

Film coated tablets containing 40 mg of the present compound and using hydroxypropyl cellulose as a water soluble macromolecular binding agent and partly pregelatinized starch and croscarmellose sodium as a disintegrant

Film coated tablets were manufactured through the manufacture of granules and uncoated tablets based on the following (1-1), (1-2), (1-3), (1-4), and (1-5) to conduct a stability test (accelerated test).

The amount added shown within the parentheses in the descriptions shows an example for preparing a formulation with the prescription shown in Example 1. In principle, other Examples can be similarly manufactured by changing the amount added by following this manufacturing method.

### (1-1) Preparation of Binding Solution (5% Aqueous Hydroxypropyl Cellulose Solution):

Hydroxypropyl cellulose (28 g) that is the water soluble macromolecular binding agent was dissolved into purified water (532 g) as a binding solution.

### (1-2) Granulation:

The present compound (200 g), mannitol (815 g), partly pregelatinized starch (280 g), and croscarmellose sodium 42 g) were added to a fluid bed granulator dryer (multiplex FD-MP-01/Powrex), and the binding solution prepared in (1-1) described above was used and sprayed to form granules under the following conditions, resulting in granule powder. After adding sodium stearyl fumarate to the resulting granule powder and mixing (40 rpm, 5 minutes), granules for tableting with prescription (B) were obtained. Sodium stearyl fumarate was mixed at an amount computed from the prescription based on the granule powder yield.

### Granulation Condition

Inlet air temperature: 80° C.

Inlet air volume: 50 to 55 m³/h

Spray rate: 10 to 15 g/min

Spray nozzle diameter: 1.2 mm

Spray pressure: 0.12 MPa

Gun position: top spray

### (1-3) Tableting:

Uncoated tablets were molded using the granules for tableting prepared in (1-2) with a rotary tableting machine HT-AP12SS-II (Hata Tekkosho).

Punch size: φ9.0 mm 13R

Thickness: 4.10 to 4.30 mm

Tableting compression force: 8 KN

### (1-4) Coating:

The uncoated tablets prepared in (1-3) were coated under the following conditions with a pan-coating apparatus FC-HICOATER-HCT-30N (Freund Corporation) so that the amount of coating would be 6.5 mg to obtain film coated tablets.

### Film Coating Conditions

Inlet air temperature: 80° C.

Inlet air volume: 0.6 m³/h

Pan rotation: 10 rpm

Spray pressure: 0.15 MPa

Liquid speed: 3 to 7 g/min

### (1-5) Accelerated Test:

The film coated tablets prepared in (1-4) were stored under the conditions described in Test Example 2.

### Example 2

Film coated tablets containing 40 mg of the present compound and using polyvinyl alcohol as a water soluble macromolecular binding agent and partly pregelatinized starch and croscarmellose sodium as a disintegrant

Film coated tablets were manufactured through the manufacture of granules and uncoated tablets based on the following (2-1), (2-2), (2-3), (2-4), and (2-5) to conduct a stability test (accelerated test).

The amount added shown within the parentheses in the descriptions shows an example for preparing a formulation with the prescription shown in Example 2. In principle, other Examples can be similarly manufactured by changing the amount added by following this manufacturing method.

### (2-1) Preparation of Binding Solution (5% Aqueous Polyvinyl Alcohol Solution):

Polyvinyl alcohol (28 g) that is the water soluble macromolecular binding agent was dissolved into purified water (532 g) as a binding solution.

### (2-2) Granulation:

The present compound (200 g), mannitol (815 g), partly pregelatinized starch (280 g), and croscarmellose sodium (42 g) were added to a fluid bed granulator dryer (multiplex FD-MP-01/Powrex), and the binding solution prepared in (2-1) described above was used and sprayed to form granules under the following conditions, resulting in granule powder. After adding sodium stearyl fumarate to the resulting granule powder and mixing (40 rpm, 5 minutes), granules for tableting with prescription (B) were obtained. Sodium stearyl fumarate was mixed at an amount computed from the prescription based on the granule powder yield.

### Granulation Condition

Inlet air temperature: 80° C.

Inlet air volume: 50 to 55 m³/h

Spray rate: 10 to 15 g/min

Spray nozzle diameter: 1.2 mm

Spray pressure: 0.12 MPa

Gun position: top spray

### (2-3) Tableting:

Uncoated tablets were molded using the granules for tableting prepared in (2-2) with a rotary tableting machine HT-AP12SS-II (Hata Tekkosho).

Punch size: φ9.0 mm 13R

Thickness: 4.10 to 4.30 mm

Tableting compression force: 8 KN

### (2-4) Coating:

The uncoated tablets prepared in (2-3) were coated under the following conditions with a pan-coating apparatus FC-HICOATER-HCT-30N (Freund Corporation) so that the amount of coating would be 6.5 mg to obtain film coated tablets.

### Film Coating Conditions

Inlet air temperature: 80° C.

Inlet air volume: 0.6 m³/h

Pan rotation: 10 rpm

Spray pressure: 0.15 MPa

Liquid speed: 3 to 7 g/min

### (2-5) Accelerated Test:

The film coated tablets prepared in (2-4) were stored under the conditions described in Test Example 2.

### Comparative Example 1

Partly pregelatinized starch free film coated tablets containing 40 mg of the present compound and using hydroxypropyl cellulose as a water soluble macromolecular binding agent and low substituted hydroxypropyl cellulose as a disintegrant

Film coated tablets were manufactured through the manufacture of granules and uncoated tablets based on the following (3-1), (3-2), (3-3), (3-4), and (3-5) to conduct a stability test (accelerated test).

The amount added shown within the parentheses in the descriptions shows an example for preparing a formulation with the prescription shown in Comparative Example 1. In principle, other Examples can be similarly manufactured by changing the amount added by following this manufacturing method.

### (3-1) Preparation of Binding Solution (5% Aqueous Hydroxypropyl Cellulose Solution):

Hydroxypropyl cellulose (28 g) that is the water soluble macromolecular binding agent was dissolved into purified water (532 g) as a binding solution.

### (3-2) Granulation:

The present compound (200 g), mannitol (927 g), and low substituted hydroxypropyl cellulose (210 g) were added to a fluid bed granulator dryer (multiplex FD-MP-01/Powrex), and the binding solution prepared in (3-1) described above was used and sprayed to form granules under the following conditions, resulting in granule powder. After adding sodium stearyl fumarate to the resulting granule powder and mixing (40 rpm, 5 minutes), granules for tableting with prescription (B) were obtained. Sodium stearyl fumarate was mixed at an amount computed from the prescription based on the granule powder yield.

### Granulation Condition

Inlet air temperature: 80° C.

Inlet air volume: 50 to 55 m³/h

Spray rate: 10 to 15 g/min

Spray nozzle diameter: 1.2 mm

Spray pressure: 0.12 MPa

Gun position: top spray

### (3-3) Tableting:

Uncoated tablets were molded using the granules for tableting prepared in (3-2) with a rotary tableting machine HT-AP12SS-II (Hata Tekkosho).

Punch size: φ9.0 mm 13R

Thickness: 4.10 to 4.30 mm

Tableting compression force: 8 KN

### (3-4) Coating:

The uncoated tablets prepared in (3-3) were coated under the following conditions with a pan-coating apparatus FC-HICOATER-HCT-30N (Freund Corporation) so that the amount of coating would be 6.5 mg to obtain film coated tablets.

### Film Coating Conditions

Inlet air temperature: 80° C.

Inlet air volume: 0.6 m³/h

Pan rotation: 10 rpm

Spray pressure: 0.15 MPa

Liquid speed: 3 to 7 g/min

### (3-5) Accelerated Test:

The film coated tablets prepared in (3-4) were stored under the conditions described in Test Example 2.

### Comparative Example 2

Film coated tablets containing 40 mg of the present compound and using hydroxypropyl cellulose as a water soluble macromolecular binding agent and partly pregelatinized starch as a disintegrant

Film coated tablets were manufactured through the manufacture of granules and uncoated tablets based on the following (4-1), (4-2), (4-3), (4-4), and (4-5) to conduct a stability test (accelerated test).

The amount added shown within the parentheses in the descriptions shows an example for preparing a formulation with the prescription shown in Comparative Example 2. In principle, other Examples can be similarly manufactured by changing the amount added by following this manufacturing method.

### (4-1) Preparation of Binding Solution (5% Aqueous Hydroxypropyl Cellulose Solution):

Hydroxypropyl cellulose (28 g) that is the water soluble macromolecular binding agent was dissolved into purified water (532 g) as a binding solution.

### (4-2) Granulation:

The present compound (200 g), mannitol (857 g), and partly pregelatinized starch (280 g) were added to a fluid bed granulator dryer (multiplex FD-MP-01/Powrex), and the binding solution prepared in (4-1) described above was used and sprayed to form granules under the following conditions, resulting in granule powder. After adding sodium stearyl fumarate to the resulting granule powder and mixing (40 rpm, 5 minutes), granules for tableting with prescription (B) were obtained. Sodium stearyl fumarate was mixed at an amount computed from the prescription based on the granule powder yield.

### Granulation Condition

Inlet air temperature: 80° C.

Inlet air volume: 50 to 55 m³/h

Spray rate: 10 to 15 g/min

Spray nozzle diameter: 1.2 mm

Spray pressure: 0.12 MPa

Gun position: top spray

### (4-3) Tableting:

Uncoated tablets were molded using the granules for tableting prepared in (4-2) with a rotary tableting machine HT-AP12SS-II (Hata Tekkosho).

Punch size: φ9.0 mm 13R

Thickness: 4.10 to 4.30 mm

Tableting compression force: 8 KN

### (4-4) Coating:

The uncoated tablets prepared in (4-3) were coated under the following conditions with a pan-coating apparatus FC-HICOATER-HCT-30N (Freund Corporation) so that the amount of coating would be 6.5 mg to obtain film coated tablets.

### Film Coating Conditions

Inlet air temperature: 80° C.

Inlet air volume: 0.6 m³/h

Pan rotation: 10 rpm

Spray pressure: 0.15 MPa

Liquid speed: 3 to 7 g/min

### (4-5) Accelerated Test:

The film coated tablets prepared in (4-4) were stored under the conditions described in Test Example 2.

### Comparative Example 3

Film coated tablets containing 40 mg of the present compound and using polyvinyl alcohol as a water soluble macromolecular binding agent and partly pregelatinized starch as a disintegrant

Film coated tablets were manufactured through the manufacture of granules and uncoated tablets based on the following (5-1), (5-2), (5-3), (5-4), and (5-5) to conduct a stability test (accelerated test).

The amount added shown within the parentheses in the descriptions shows an example for preparing a formulation with the prescription shown in Comparative Example 3. In principle, other Examples can be similarly manufactured by changing the amount added by following this manufacturing method.

### (5-1) Preparation of Binding Solution (5% Aqueous Polyvinyl Alcohol Solution):

Polyvinyl alcohol (28 g) that is the water soluble macromolecular binding agent was dissolved into purified water (532 g) as a binding solution.

### (5-2) Granulation:

The present compound (200 g), mannitol (857 g), and partly pregelatinized starch (280 g) were added to a fluid bed granulator dryer (multiplex FD-MP-01/Powrex), and the binding solution prepared in (5-1) described above was used and sprayed to form granules under the following conditions, resulting in granule powder. After adding sodium stearyl fumarate to the resulting granule powder and mixing (40 rpm, 5 minutes), granules for tableting with prescription (B) were obtained. Sodium stearyl fumarate was mixed at an amount computed from the prescription based on the granule powder yield.

### Granulation Condition

Inlet air temperature: 80° C.

Inlet air volume: 50 to 55 m³/h

Spray rate: 10 to 15 g/min

Spray nozzle diameter: 1.2 mm

Spray pressure: 0.12 MPa

Gun position: top spray

### (5-3) Tableting:

Uncoated tablets were molded using the granules for tableting prepared in (5-2) with a rotary tableting machine HT-AP12SS-II (Hata Tekkosho).

Punch size: φ9.0 mm 13R

Thickness: 4.10 to 4.30 mm

Tableting compression force: 8 KN

### (5-4) Coating:

The uncoated tablets prepared in (5-3) were coated under the following conditions with a pan-coating apparatus FC-HICOATER-HCT-30N (Freund Corporation) so that the amount of coating would be 6.5 mg to obtain film coated tablets.

### Film Coating Conditions

Inlet air temperature: 80° C.

Inlet air volume: 0.6 m³/h

Pan rotation: 10 rpm

Spray pressure: 0.15 MPa

Liquid speed: 3 to 7 g/min

### (5-5) Accelerated Test:

The film coated tablets prepared in (5-4) were stored under the conditions described in Test Example 2.

(A) Prescription of granule powder (prescription of granules of Examples 1 and 2 and Comparative Examples 1, 2, and 3)

TABLE 1

| | | | | | Comparative | | Comparative | | Comparative | |
|---|---|---|---|---|---|---|---|---|---|---|
| | Example 1 | | Example 2 | | Example 1 | | Example 2 | | Example 3 | |
| Present compound | 40 | mg | 40 | mg | 40 | mg | 40 | mg | 40 | mg |
| Mannitol | 163.0 | mg | 163.0 | mg | 185.4 | mg | 171.4 | mg | 171.4 | mg |

| | | | | | | | | | |
|---|---|---|---|---|---|---|---|---|---|
| Partly | 56.0 | mg | 56.0 | mg | — | 56.0 | mg | 56.0 | mg |
| pregelatinized | | | | | | | | | |
| starch | | | | | | | | | |

| | | | | | | | |
|---|---|---|---|---|---|---|---|
| Croscarmellose | 8.4 | mg | 8.4 | mg | — | — | — |
| sodium | | | | | | | |

| Low substituted | — | | — | 42.0 | mg | — | | — |
|---|---|---|---|---|---|---|---|---|
| hydroxypropyl | | | | | | | | |
| cellulose | | | | | | | | |
| Hydroxypropyl | 5.6 | mg | — | 5.6 | mg | 5.6 | mg | — |
| cellulose | | | | | | | | |

| | | | | | | | |
|---|---|---|---|---|---|---|---|
| Polyvinyl alcohol | — | 5.6 | mg | — | — | 5.6 | mg |

(B) Prescription of granules for tableting/uncoated tablets (prescription of granules for tableting/uncoated tablets of Examples 1 and 2 and Comparative Examples 1, 2, and 3)

TABLE 2

| | Example | | Example | | Comparative | | Comparative | | Comparative | |
|---|---|---|---|---|---|---|---|---|---|---|
| | 1 | | 2 | | Example 1 | | Example 2 | | Example 3 | |
| Granules of | 273.0 | mg | 273.0 | mg | 273.0 | mg | 273.0 | mg | 273.0 | mg |
| (A) | | | | | | | | | | |
| Sodium | 7.0 | mg | 7.0 | mg | 7.0 | mg | 7.0 | mg | 7.0 | mg |
| stearyl | | | | | | | | | | |
| fumarate | | | | | | | | | | |

(C) Prescription of film coated tablets (prescription of film coated tablets of Examples 1 and 2 and Comparative Examples 1, 2, and 3)

TABLE 3

| | Example | | Example | | Comparative | | Comparative | | Comparative | |
|---|---|---|---|---|---|---|---|---|---|---|
| | 1 | | 2 | | Example 1 | | Example 2 | | Example 3 | |
| Uncoated | 280.0 | mg | 280.0 | mg | 280.0 | mg | 280.0 | mg | 280.0 | mg |
| tablets of | | | | | | | | | | |
| (B) | | | | | | | | | | |
| Hypromellose | 4.68 | mg | 4.68 | mg | 4.68 | mg | 4.68 | mg | 4.68 | mg |
| Titanium | 0.884 | mg | 0.884 | mg | 0.884 | mg | 0.884 | mg | 0.884 | mg |
| oxide | | | | | | | | | | |
| Propylene | 0.884 | mg | 0.884 | mg | 0.884 | mg | 0.884 | mg | 0.884 | mg |
| glycol | | | | | | | | | | |
| Yellow iron | 0.052 | mg | 0.052 | mg | 0.052 | mg | 0.052 | mg | 0.052 | mg |
| sesquioxide | | | | | | | | | | |

The quality of formulations obtained by the method described above was evaluated by the following method.

Dissolution Test

A dissolution test was conducted in accordance with the Japanese Pharmacopoeia, Dissolution Test, Paddle Method (Apparatus 2). The measurement conditions are shown below.

Test solution: 2^{nd} fluid for dissolution test (pH 6.8)

Paddle rotations: 50 rpm

Test solution: 900 ml

### Test Example 1

Dissolution Test

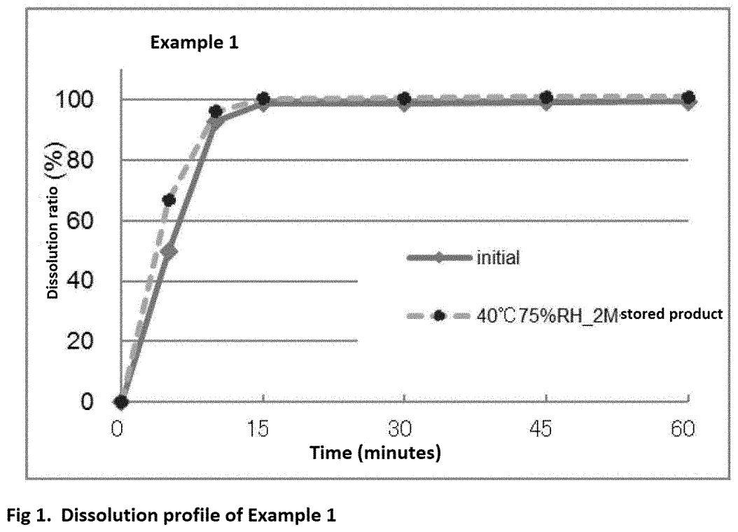

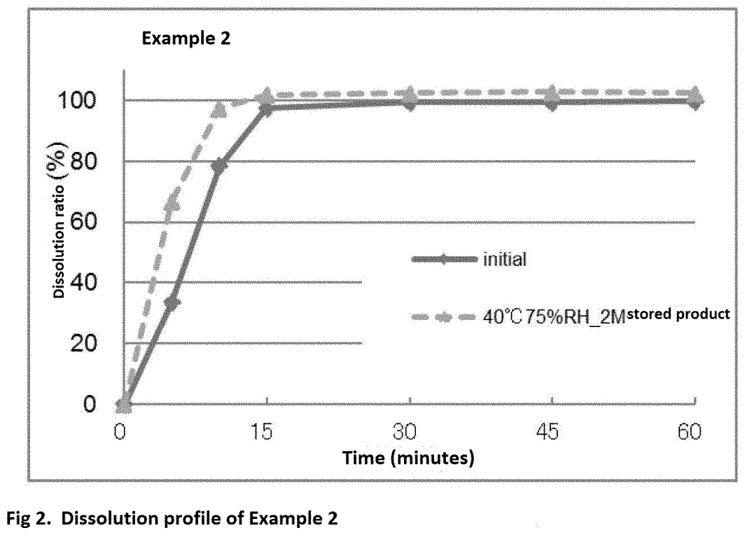

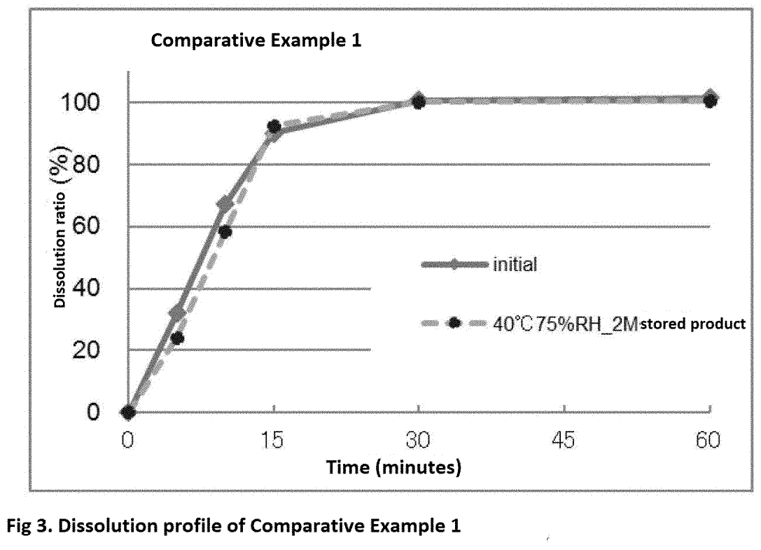

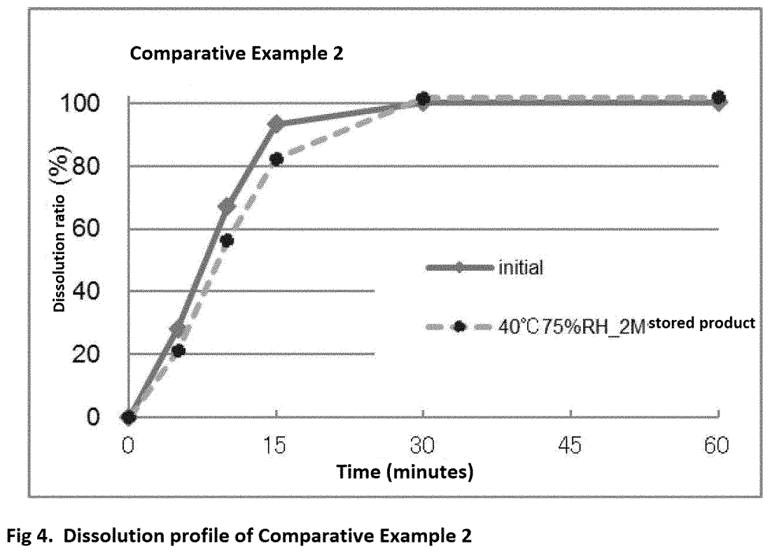

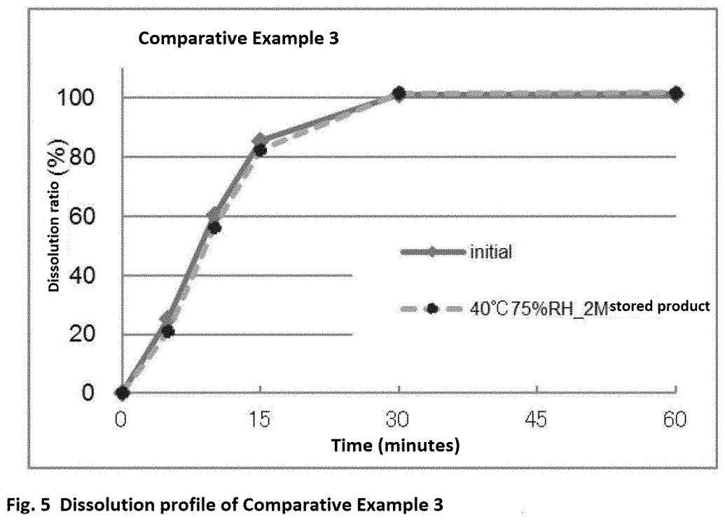

Dissolution tests were conducted on the film coated tablets of Examples 1 and 2 and Comparative Examples 1, 2, and 3, and stored products (accelerated test product) of Examples 1 and 2 and Comparative Examples 1, 2, and 3 stored under the storage conditions described in Test Example 2. The dissolution ratios (%) are shown in Tables 4 and 5. The dissolution profiles of Examples 1 and 2 and Comparative Examples 1, 2, and 3 are shown in FIGS. 1 to 5.

### Test Example 2

Accelerated Test

Hygrostat incubator conditions were set to 40° C./75% RH. Prepared film coated tablets were placed in an HDPE bottle (material: high-density polyethylene (HDPE), volume: 30 ml, H: 61 mm, W: 35 mm, L: 30 mm)) and stored for 2 months in a hygrostat incubator in an open state.

TABLE 4

| Before | Example 1 | Example 2 | Comparative | Comparative | Comparative |
|---|---|---|---|---|---|
| accelerated | film | film | Example 1 | Example 2 | Example 3 |
| storage | coated | coated | film coated | film coated | film coated |
| (Initial) | tablet | tablet | tablet | tablet | tablet |
| 5 minute | 49.8 | 33.4 | 32.0 | 28.3 | 34.2 |
| dissolution | | | | | |
| ratio | | | | | |
| 10 minute | 92.9 | 78.3 | 67.3 | 67.0 | 73.8 |
| dissolution | | | | | |
| ratio | | | | | |
| 15 minute | 98.8 | 97.3 | 90.1 | 93.3 | 96.2 |
| dissolution | | | | | |
| ratio | | | | | |

TABLE 5

| After | | | | | |
|---|---|---|---|---|---|
| accelerated | | | Comparative | Comparative | Comparative |
| storage | Example 1 | Example 2 | Example 1 | Example 2 | Example 3 |
| (40° C./75% | accelerated | accelerated | accelerated | accelerated | accelerated |
| RH_2M stored | test | test | test | test | test |
| product) | product | product | product | product | product |
| 5 minute | 66.8 | 66.6 | 24.1 | 21.2 | 25.4 |
| dissolution | | | | | |
| ratio | | | | | |
| 10 minute | 96.2 | 97.4 | 58.4 | 56.3 | 60.2 |
| dissolution | | | | | |
| ratio | | | | | |
| 15 minute | 100.4 | 101.8 | 92.4 | 82.4 | 85.5 |
| dissolution | | | | | |
| ratio | | | | | |

In view of Tables 4 and 5, Examples 1 and 2 comprising partly pregelatinized starch and croscarmellose sodium (cellulose-based disintegrant) as a disintegrant exhibited a better dissolution property than Comparative Examples 1, 2, and 3 in terms of the dissolution ratio of accelerated test products.

### Example 3

Film coated tablets comprising 5 mg of the present compound

Film coated tablets were manufactured through the manufacture of granules and uncoated tablets based on the following (6-1), (6-2), (6-3), (6-4), and (6-5) to conduct a stability test (accelerated test).

The amount added shown within the parentheses in the descriptions shows an example for preparing a formulation with the prescription shown in Example 3. In principle, other Examples can be similarly manufactured by changing the amount added by following this manufacturing method.

### (6-1) Preparation of Binding Solution (5% Aqueous Hydroxypropyl Cellulose Solution):

Hydroxypropyl cellulose (21 g) that is the water soluble macromolecular binding agent was dissolved into purified water (399 g) as a binding solution.

### (6-2) Granulation:

The present compound (75 g), mannitol (686.25 g), partly pregelatinized starch (210 g), and croscarmellose sodium (31.5 g) were added to a fluid bed granulator dryer (multiplex FD-MP-01/Powrex), and the binding solution prepared in (6-1) described above was used and sprayed to form granules under the following conditions, resulting in granule powder. After adding sodium stearyl fumarate to the resulting granule powder and mixing (40 rpm, 5 minutes), granules for tableting with prescription (E) were obtained. Sodium stearyl fumarate was mixed at an amount computed from the prescription based on the granule powder yield.

### Granulation Condition

Inlet air temperature: 80° C.

Inlet air volume: 50 m³/H

Spray rate: 10 g/min

Spray nozzle diameter: 1.2 mm

Spray pressure: 0.12 MPa

Gun position: top spray

### (6-3) Tableting:

Uncoated tablets were molded using the granules for tableting prepared in (6-2) with a rotary tableting machine VEL2 0308SW2MZ model (Kikusui Seisakusho).

Punch size: φ5.5 mm 7R

Thickness: 2.70 to 3.00 mm

Tableting compression force: 5 KN

### (6-4) Coating:

The uncoated tablets prepared in (6-3) were coated under the following conditions with a HICOATER-HC-LABO (Freund Corporation) so that the amount of coating would be 2.5 mg to obtain film coated tablets.

### Film Coating Conditions

Inlet air temperature: 70° C.

Inlet air volume: 0.5 m³/min

Pan rotation: 20 rpm

Spray pressure: 0.15 MPa

Liquid speed: 3.5 g/min

### (6-5) Accelerated Test:

The film coated tablets prepared in (6-4) were stored under the conditions described in Test Example 4.

### Example 4

Film coated tablets comprising 10 mg of the present compound

Film coated tablets were manufactured through the manufacture of granules and uncoated tablets based on the following (7-1), (7-2), (7-3), (7-4), and (7-5) to conduct a stability test (accelerated test).

The amount added shown within the parentheses in the descriptions shows an example for preparing a formulation with the prescription shown in Example 4. In principle, other Examples can be similarly manufactured by changing the amount added by following this manufacturing method.

### (7-1) Preparation of Binding Solution (59 Aqueous Hydroxypropyl Cellulose Solution):

Hydroxypropyl cellulose (21 g) that is the water soluble macromolecular binding agent was dissolved into purified water (399 g) as a binding solution.

### (7-2) Granulation:

The present compound (150 g), mannitol (611.25 g), partly pregelatinized starch (210 g), and croscarmellose sodium (31.5 g) were added to a fluid bed granulator dryer (multiplex FD-MP-01/Powrex), and the binding solution prepared in (7-1) described above was used and sprayed to form granules under the following conditions, resulting in granule powder. After adding sodium stearyl fumarate to the resulting granule powder and mixing (40 rpm, 5 minutes), granules for tableting with prescription (E) were obtained. Sodium stearyl fumarate was mixed at an amount computed from the prescription based on the granule powder yield.

### Granulation Condition

Inlet air temperature: 80° C.

Inlet air volume: 50 m³/H

Spray rate: 10 g/min

Spray nozzle diameter: 1.2 mm

Spray pressure: 0.12 MPa

Gun position: top spray

### (7-3) Tableting:

Uncoated tablets were molded using the granules for tableting prepared in (7-2) with a rotary tableting machine VEL2 0308SW2MZ model (Kikusui Seisakusho).

Punch size: φ5.5 mm 7R

Thickness: 2.70 to 3.00 mm

Tableting compression force: 5 KN

### (7-4) Coating:

The uncoated tablets prepared in (7-3) were coated under the following conditions with a HICOATER-HC-LABO (Freund Corporation) so that the amount of coating would be 2.5 mg to obtain film coated tablets.

### Film Coating Conditions

Inlet air temperature: 70° C.

Inlet air volume: 0.5 m³/min

Pan rotation: 20 rpm

Spray pressure: 0.15 MPa

Liquid speed: 3.5 g/min

### (7-5) Accelerated Test:

The film coated tablets prepared in (7-4) were stored under the conditions described in Test Example 4.

### Example 5

Film coated tablets comprising 20 mg of the present compound

Film coated tablets were manufactured through the manufacture of granules and uncoated tablets based on the following (8-1), (8-2), (8-3), (8-4), and (8-5) to conduct a stability test (accelerated test).

The amount added shown within the parentheses in the descriptions shows an example for preparing a formulation with the prescription shown in Example 5. In principle, other Examples can be similarly manufactured by changing the amount added by following this manufacturing method.

### (8-1) Preparation of Binding Solution (5% Aqueous Hydroxypropyl Cellulose Solution):

Hydroxypropyl cellulose (21 g) that is the water soluble macromolecular binding agent was dissolved into purified water (399 g) as a binding solution.

### (8-2) Granulation:

The present compound (150 g), mannitol (611.25 g), partly pregelatinized starch (210 g), and croscarmellose sodium (31.5 g) were added to a fluid bed granulator dryer (multiplex FD-MP-O1/Powrex), and the binding solution prepared in (8-1) described above was used and sprayed to form granules under the following conditions, resulting in granule powder. After adding sodium stearyl fumarate to the resulting granule powder and mixing (40 rpm, 5 minutes), granules for tableting with prescription (E) were obtained. Sodium stearyl fumarate was mixed at an amount computed from the prescription based on the granule powder yield.

### Granulation Condition

Inlet air temperature: 80° C.

Inlet air volume: 50 m³/H

Spray rate: 10 g/min

Spray nozzle diameter: 1.2 mm

Spray pressure: 0.12 MPa

Gun position: top spray

### (8-3) Tableting:

Uncoated tablets were molded using the granules for tableting prepared in (8-2) with a rotary tableting machine VEL2 0308SW2MZ model (Kikusui Seisakusho).

Punch size: φ7.0 mm 10R

Thickness: 3.20 to 3.60 mm

Tableting compression force: 7 KN

### (8-4) Coating:

The uncoated tablets prepared in (8-3) were coated under the following conditions with a HICOATER-HC-LABO (Freund Corporation) so that the amount of coating would be 4 mg to obtain film coated tablets.

### Film Coating Conditions

Inlet air temperature: 70° C.

Inlet air volume: 0.5 m³/min

Pan rotation: 20 rpm

Spray pressure: 0.15 MPa

Liquid speed: 3.5 g/min

### (8-5) Accelerated Test:

The film coated tablets prepared in (8-4) were stored under the conditions described in Test Example 4.

### Example 6

Film coated tablets containing 40 mg of the present compound, and partly pregelatinized starch and low substituted hydroxypropyl cellulose as a disintegrant

Film coated tablets were manufactured through the manufacture of granules and uncoated tablets based on the following (9-1), (9-2), (9-3), (9-4), and (9-5) to conduct a stability test (accelerated test).

The amount added shown within the parentheses in the descriptions shows an example for preparing a formulation with the prescription shown in Example 6. In principle, other Examples can be similarly manufactured by changing the amount added by following this manufacturing method.

### (9-1) Preparation of Binding Solution (5% Aqueous Hydroxypropyl Cellulose Solution):

Hydroxypropyl cellulose (28 g) that is the water soluble macromolecular binding agent was dissolved into purified water (532 g) as a binding solution.

### (9-2) Granulation:

The present compound (200 g), mannitol (647.00 g), partly pregelatinized starch (280.0 g), and low substituted hydroxypropyl cellulose (210.0 g) were added to a fluid bed granulator dryer (multiplex FD-MP-01/Powrex), and the binding solution prepared in (9-1) described above was used and sprayed to form granules under the following conditions, resulting in granule powder. After adding sodium stearyl fumarate to the resulting granule powder and mixing (40 rpm, 5 minutes), granules for tableting with prescription (E) were obtained. Sodium stearyl fumarate was mixed at an amount computed from the prescription based on the granule powder yield.

### Granulation Condition

Inlet air temperature: 80° C.

Inlet air volume: 50 m³/H

Spray rate: 10 g/min

Spray nozzle diameter: 1.2 mm

Spray pressure: 0.12 MPa

Gun position: top spray

### (9-3) Tableting:

Uncoated tablets were molded using the granules for tableting prepared in (9-2) with a rotary tableting machine VEL2 0308SW2MZ model (Kikusui Seisakusho).

Punch size: φ9.0 mm 13R

Thickness: 4.00 to 4.50 mm

Tableting compression force: 7 KN

### (9-4) Coating:

The uncoated tablets prepared in (9-3) were coated under the following conditions with a HICOATER-HC-LABO (Freund Corporation) so that the amount of coating would be 6.5 mg to obtain film coated tablets.

### Film Coating Conditions

Inlet air temperature: 70° C.

Inlet air volume: 0.5 m³/min

Pan rotation: 20 rpm

Spray pressure: 0.15 MPa

Liquid speed: 3.5 g/min

### (9-5) Accelerated Test:

The film coated tablets prepared in (9-4) were stored under the conditions described in Test Example 4.

### Example 7

Film coated tablets containing 40 mg of the present compound and comprising hydroxypropyl cellulose as a water soluble macromolecular binding agent and croscarmellose sodium as a disintegrant

Film coated tablets were manufactured through the manufacture of granules and uncoated tablets based on the following (10-1), (10-2), (10-3), (10-4), and (10-5) to conduct a stability test (accelerated test).

The amount added shown within the parentheses in the descriptions shows an example for preparing a formulation with the prescription shown in Example 7. In principle, other Examples can be similarly manufactured by changing the amount added by following this manufacturing method.

### (10-1) Preparation of Binding Solution (5% Aqueous Hydroxypropyl Cellulose Solution):

Hydroxypropyl cellulose (28 g) that is the water soluble macromolecular binding agent was dissolved into purified water (532 g) as a binding solution.

### (10-2) Granulation:

The present compound (200 g), mannitol (1095.00 g), and croscarmellose sodium (42.0 g) were added to a fluid bed granulator dryer (multiplex FD-MP-01/Powrex), and the binding solution prepared in (10-1) described above was used and sprayed to form granules under the following conditions, resulting in granule powder. After adding sodium stearyl fumarate to the resulting granule powder and mixing (40 rpm, 5 minutes), granules for tableting with prescription (E) were obtained. Sodium stearyl fumarate was mixed at an amount computed from the prescription based on the granule powder yield.

### Granulation Condition

Inlet air temperature: 80° C.

Inlet air volume: 50 m³/H

Spray rate: 10 g/min

Spray nozzle diameter: 1.2 mm

Spray pressure: 0.12 MPa

Gun position: top spray

### (10-3) Tableting:

Uncoated tablets were molded using the granules for tableting prepared in (10-2) with a rotary tableting machine VEL2 0308SW2MZ model (Kikusui Seisakusho).

Punch size: φ9.0 mm 13R

Thickness: 4.00 to 4.50 mm

Tableting compression force: 7 KN

### (10-4) Coating:

The uncoated tablets prepared in (10-3) were coated under the following conditions with a HICOATER-HC-LABO (Freund Corporation) so that the amount of coating would be 6.5 mg to obtain film coated tablets.

### Film Coating Conditions

Inlet air temperature: 70° C.

Inlet air volume: 0.5 m³/min

Pan rotation: 20 rpm

Spray pressure: 0.15 MPa

Liquid speed: 3.5 g/min

### (10-5) Accelerated Test:

The film coated tablets prepared in (10-4) were stored under the conditions described in Test Example 4.

### Example 8

Film coated tablets containing 40 mg of the present compound, and comprising polyvinyl alcohol as a water soluble macromolecular binding agent and croscarmellose sodium as a disintegrant

Film coated tablets were manufactured through the manufacture of granules and uncoated tablets based on the following (11-1), (11-2), (11-3), (11-4), and (11-5) to conduct a stability test (accelerated test).

The amount added shown within the parentheses in the descriptions shows an example for preparing a formulation with the prescription shown in Comparative Example 4. In principle, other Examples can be similarly manufactured by changing the amount added by following this manufacturing method.

### (11-1) Preparation of Binding Solution (5% Aqueous Polyvinyl Alcohol Solution):

Polyvinyl alcohol (28 g) that is the water soluble macromolecular binding agent was dissolved into purified water (532 g) as a binding solution.

### (11-2) Granulation:

The present compound (200 g), mannitol (1095.00 g), and croscarmellose sodium (42.0 g) were added to a fluid bed granulator dryer (multiplex FD-MP-01/Powrex), and the binding solution prepared in (11-1) described above was used and sprayed to form granules under the following conditions, resulting in granule powder. After adding sodium stearyl fumarate to the resulting granule powder and mixing (40 rpm, 5 minutes), granules for tableting with prescription (E) were obtained. Sodium stearyl fumarate was mixed at an amount computed from the prescription based on the granule powder yield.

### Granulation Condition

Inlet air temperature: 80° C.

Inlet air volume: 50 m³/H

Spray rate: 10 g/min

Spray nozzle diameter: 1.2 mm

Spray pressure: 0.12 MPa

Gun position: top spray

### (11-3) Tableting:

Uncoated tablets were molded using the granules for tableting prepared in (11-2) with a rotary tableting machine VEL2 0308SW2MZ model (Kikusui Seisakusho).

Punch size: φ9.0 mm 13R

Thickness: 4.00 to 4.50 mm

Tableting compression force: 7 KN

### (11-4) Coating:

The uncoated tablets prepared in (11-3) were coated under the following conditions with a HICOATER-HC-LABO (Freund Corporation) so that the amount of coating would be 6.5 mg to obtain film coated tablets.

### Film Coating Conditions

Inlet air temperature: 70° C.

Inlet air volume: 0.5 m³/min

Pan rotation: 20 rpm

Spray pressure: 0.15 MPa

Liquid speed: 3.5 g/min

### (11-5) Accelerated Test:

The film coated tablets prepared in (11-4) were stored under the conditions described in Test Example 4.

(D) Prescription of granule powder (prescription of granules of Examples 3, 4, 5, 5, 7, and 8)

TABLE 6

| | Example | | Example | | Example | | Example | | Example | | Example | |
|---|---|---|---|---|---|---|---|---|---|---|---|---|
| | 3 | | 4 | | 5 | | 6 | | 7 | | 8 | |
| Present | 5 | mg | 10 | mg | 20 | mg | 40 | mg | 40 | mg | 40 | mg |
| compound | | | | | | | | | | | | |
| Mannitol | 45.75 | mg | 40.75 | mg | 81.50 | mg | 129.4 | mg | 219.0 | mg | 219.0 | mg |

| | | | | | | | | | | |
|---|---|---|---|---|---|---|---|---|---|---|
| Partly | 14.0 | mg | 14.0 | mg | 28.0 | mg | 56.0 | mg | — | — |
| pregelatinized | | | | | | | | | | |
| starch | | | | | | | | | | |

| | | | | | | | | | | | |
|---|---|---|---|---|---|---|---|---|---|---|---|
| Croscarmellose | 2.1 | mg | 2.1 | mg | 4.2 | mg | — | 8.4 | mg | 8.4 | mg |
| sodium | | | | | | | | | | | |

| Low substituted | — | | — | | — | | 42.0 | mg | — | | — |
|---|---|---|---|---|---|---|---|---|---|---|---|
| hydroxypropyl | | | | | | | | | | | |
| cellulose | | | | | | | | | | | |
| Hydroxypropyl | 1.4 | mg | 1.4 | mg | 2.8 | mg | 5.6 | mg | 5.6 | mg | — |
| cellulose | | | | | | | | | | | |

| | | | | | | | |
|---|---|---|---|---|---|---|---|
| Polyvinyl | — | — | — | — | — | 5.6 | mg |
| alcohol | | | | | | | |

(E) Prescription of granules for tableting/uncoated tablets (prescription of granules for tableting/uncoated tablets of Examples 3, 4, 5, 5, 7, and 8)

TABLE 7

| | Example | | Example | | Example | | Example | | Example | | Example | |
|---|---|---|---|---|---|---|---|---|---|---|---|---|
| | 3 | | 4 | | 5 | | 6 | | 7 | | 8 | |
| Granules | 68.25 | mg | 68.25 | mg | 136.5 | mg | 273.0 | mg | 273.0 | mg | 273.0 | mg |
| of (D) | | | | | | | | | | | | |
| Sodium | 1.75 | mg | 1.75 | mg | 3.5 | mg | 7.0 | mg | 7.0 | mg | 7.0 | mg |
| stearyl | | | | | | | | | | | | |
| fumarate | | | | | | | | | | | | |

(F) Prescription of film coated tablets (prescription of film coated tablets of Examples 3, 4, 5, 5, 7, and 8)

TABLE 8

| | Example | | Example | | Example | | Example | | Example | | Example | |
|---|---|---|---|---|---|---|---|---|---|---|---|---|
| | 3 | | 4 | | 5 | | 6 | | 7 | | 8 | |
| Uncoated | 70.0 | mg | 70.0 | mg | 140.0 | mg | 280.0 | mg | 280.0 | mg | 280.0 | mg |
| tablet of | | | | | | | | | | | | |
| (E) | | | | | | | | | | | | |
| Hypromellose | 1.80 | mg | 1.80 | mg | 2.88 | mg | 4.68 | mg | 4.68 | mg | 4.68 | mg |
| Titanium | 0.34 | mg | 0.34 | mg | 0.544 | mg | 0.884 | mg | 0.884 | mg | 0.884 | mg |
| oxide | | | | | | | | | | | | |
| Propylene | 0.34 | mg | 0.34 | mg | 0.544 | mg | 0.884 | mg | 0.884 | mg | 0.884 | mg |
| glycol | | | | | | | | | | | | |
| Yellow iron | 0.02 | mg | 0.02 | mg | 0.032 | mg | 0.052 | mg | 0.052 | mg | 0.052 | mg |
| sesquioxide | | | | | | | | | | | | |

The quality of the formulations obtained by the method described above was evaluated by the following method.

Dissolution Test

A dissolution test was conducted in accordance with the Japanese Pharmacopoeia, Dissolution Test, Paddle Method (Apparatus 2). The measurement conditions are shown below.

Test solution: 2^{nd} fluid for dissolution test (pH 6.8)

Paddle rotations: 50 rpm

Test solution: 900 ml

### Test Example 3

Dissolution Test

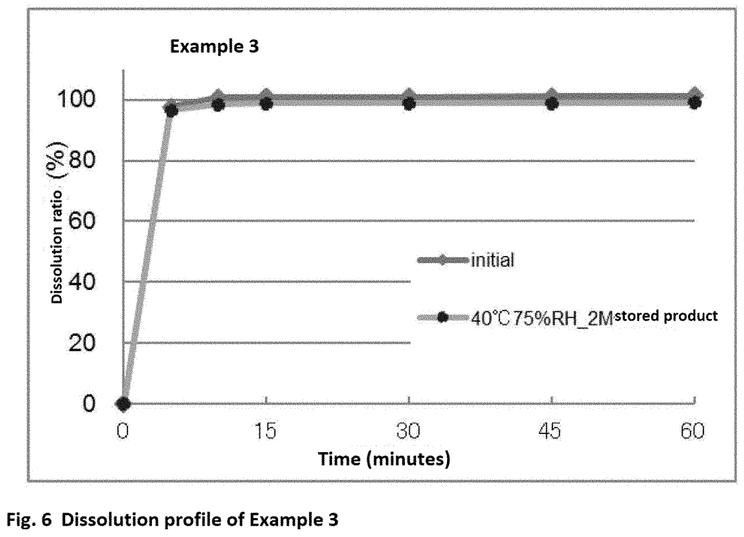

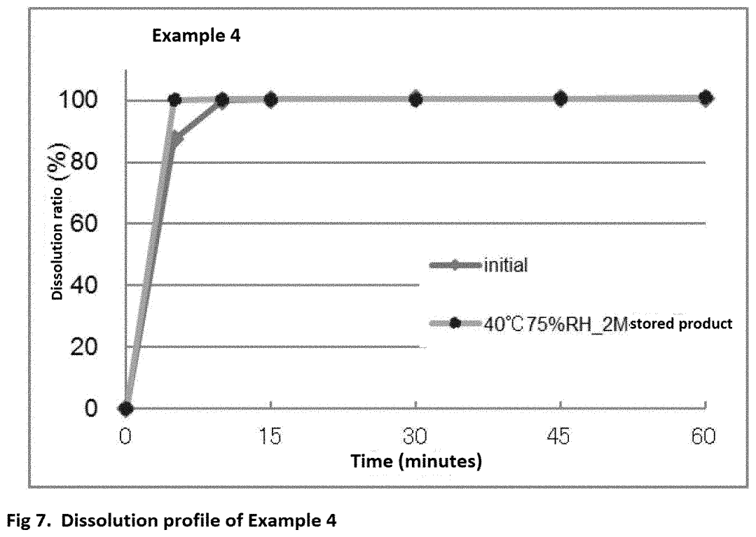

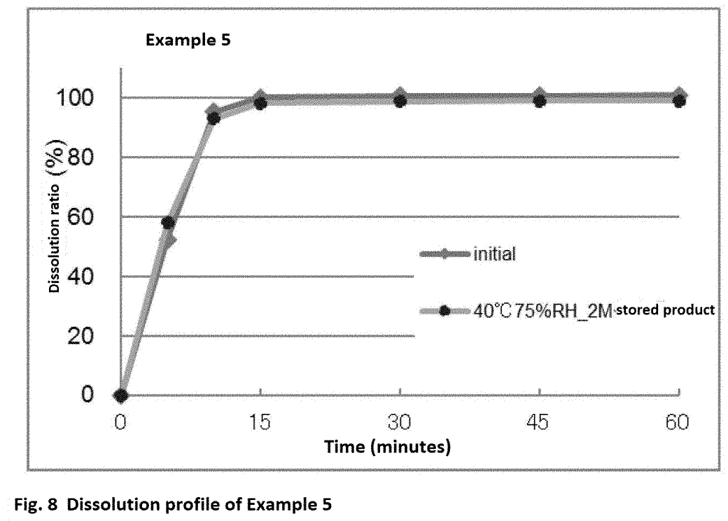

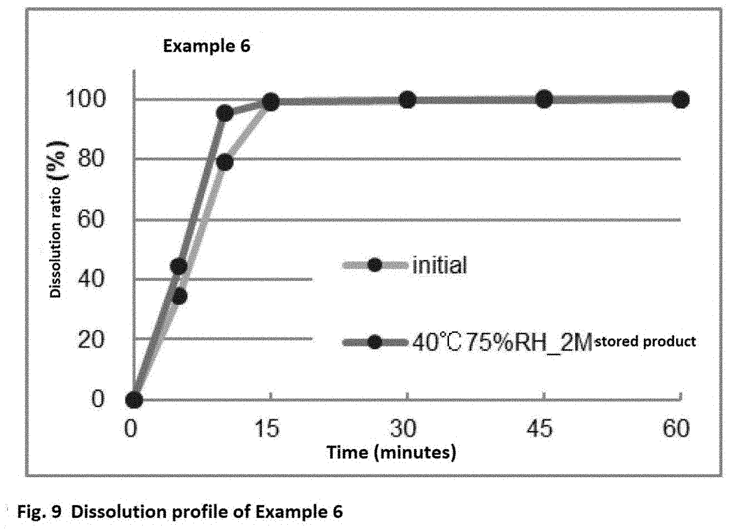

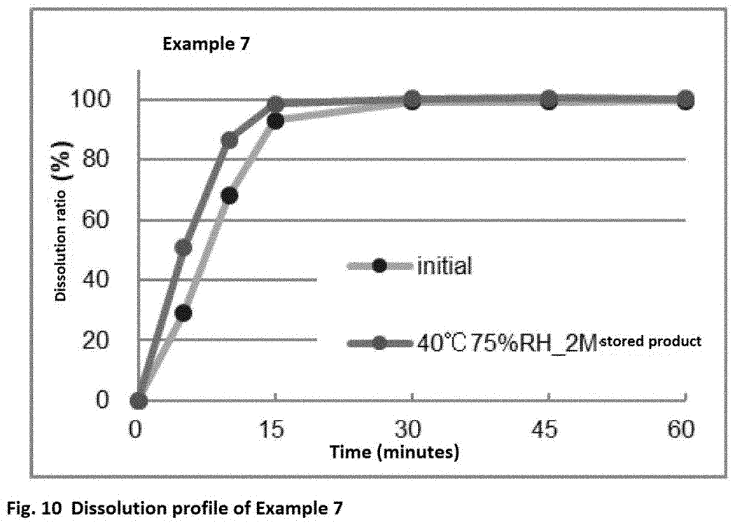

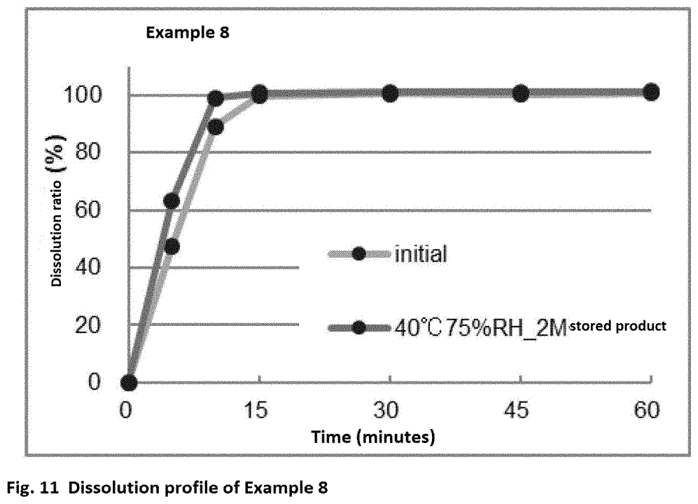

Dissolution tests were conducted on the film coated tablets of Examples 3, 4, 5, 6, 7, and 8 and stored products (accelerated test product) of Examples 3, 4, 5, 6, 7, and 8 stored under the storage conditions described in Test Example 4. The dissolution ratios (%) are shown in Tables 9 and 10. The dissolution profiles of Examples 3, 4, 5, 6, 7, and 8 are shown in FIGS. 6 to 11.

### Test Example 4

Accelerated Test

Hygrostat incubator conditions were set to 40° C./75% RH. Prepared film coated tablets were placed in an HDPE bottle (material: high-density polyethylene (HDPE), volume: 30 ml, H: 61 mm, W: 35 mm, L: 30 mm)) and stored for 2 months in a hygrostat incubator in an open state.

TABLE 9

| Before | Example 3 | Example 4 | Example 5 | Example 6 | Example 7 | Example 8 |
|---|---|---|---|---|---|---|
| accelerated | film | film | film | film | film | film |
| storage | coated | coated | coated | coated | coated | coated |
| (Initial) | tablet | tablet | tablet | tablet | tablet | tablet |
| 5 minute | 97.6 | 87.4 | 52.3 | 34.6 | 29.3 | 47.6 |
| dissolution | | | | | | |
| ratio | | | | | | |
| 10 minute | 100.7 | 99.9 | 95.3 | 79.0 | 68.0 | 89.1 |
| dissolution | | | | | | |
| ratio | | | | | | |
| 15 minute | 100.9 | 100.3 | 100.1 | 99.0 | 93.0 | 99.7 |
| dissolution | | | | | | |
| ratio | | | | | | |

TABLE 10

| After | | | | | | |
|---|---|---|---|---|---|---|
| accelerated | | | | | | |
| storage | | | | | | |
| (40° C./75% | Example 3 | Example 4 | Example 5 | Example 6 | Example 7 | Example 8 |
| RH_2M stored | accelerated | accelerated | accelerated | accelerated | accelerated | accelerated |
| product) | test product | test product | test product | test product | test product | test product |
| 5 minute | 96.5 | 100.1 | 58.0 | 44.2 | 50.8 | 63.3 |
| dissolution | | | | | | |
| ratio | | | | | | |
| 10 minute | 98.5 | 100.3 | 93.1 | 95.3 | 86.7 | 99.0 |
| dissolution | | | | | | |
| ratio | | | | | | |
| 15 minute | 98.7 | 100.3 | 98.2 | 99.2 | 98.6 | 100.8 |
| dissolution | | | | | | |
| ratio | | | | | | |

In view of Tables 9 and 10, Examples 3, 4, and 5 comprising partly pregelatinized starch and croscarmellose sodium (cellulose-based disintegrant) as a disintegrant exhibited an excellent dissolution property, even after heated and humidified storage, at any drug content.

Example 6 comprising partly pregelatinized starch and low substituted hydroxypropyl cellulose (cellulose-based disintegrant) as a disintegrant exhibited an excellent dissolution property, even after heated and humidified storage.

Examples 7 and 8 comprising only croscarmellose sodium (cellulose-based disintegrant) as a disintegrant exhibited an excellent dissolution property, regardless of the water soluble macromolecular binding agent.

As disclosed above, the present disclosure is exemplified by the use of its preferred embodiments. However, it is understood that the scope of the present disclosure should be interpreted based solely on the Claims. It is also understood that any patent, any patent application, and any other references cited herein should be incorporated herein by reference in the same manner as the contents are specifically described herein.

### INDUSTRIAL APPLICABILITY

The present disclosure can provide a solid oral formulation comprising (i) (S)-4-amino-5-chloro-N-[{4-[(1-hydroxyacetyl-4-piperidinyl)methyl]-2-morpholinyl}methyl]-2-methoxybenzamide (present compound) or a pharmaceutically acceptable salt thereof, or a hydrate or solvate thereof, as an active ingredient, ii) a disintegrant, and (iii) a water soluble macromolecular binding agent, wherein the formulation is capable of stably releasing the active ingredient, even after heated or humidified storage, for a desired period of time.

1. A solid oral formulation comprising:
(i) (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof;
(ii) a disintegrant; and
(iii) a water-soluble macromolecular binding agent.

2. The solid oral formulation of claim 1, wherein the disintegrant comprises a cellulose-based disintegrant.

3. The solid oral formulation of claim 1 or 2, wherein the disintegrant comprises croscarmellose sodium or a combination of low substituted hydroxypropyl cellulose and pregelatinized starch.

4. The solid oral formulation of any one of claims 1 to 3, wherein the disintegrant comprises croscarmellose sodium.

5. The solid oral formulation of any one of claims 1 to 4, wherein the disintegrant comprises pregelatinized starch.

6. The solid oral formulation of any one of claims 1 to 5, wherein the disintegrant comprises a combination of croscarmellose sodium and pregelatinized starch.

7. The solid oral formulation of any one of claims 1 to 6, wherein the disintegrant comprises a combination of low substituted hydroxypropyl cellulose and pregelatinized starch.

8. The solid oral formulation of any one of claims 1 to 7, wherein the water soluble macromolecular binding agent is selected from the group consisting of polyvinylpyrrolidone, copolyvidone, hydroxypropyl cellulose, and polyvinyl alcohol.

9. The solid oral formulation of any one of claims 1 to 8, wherein the water soluble macromolecular binding agent is selected from the group consisting of hydroxypropyl cellulose and polyvinyl alcohol.

10. The solid oral formulation of any one of claims 3 to 9, wherein a portion of the pregelatinized starch that is soluble in cold water is 40% by weight or less.

11. The solid oral formulation of any one of claims 3 to 9, wherein a portion of the pregelatinized starch that is soluble in water is 40% by weight or less.

12. The solid oral formulation of any one of claims 1 to 11, wherein content of the disintegrant is 1 to 50% by weight with respect to 100% by weight of the formulation.

13. The solid oral formulation of any one of claims 1 to 12, wherein content of the disintegrant other than the pregelatinized starch is 1 to 10% by weight with respect to 100% by weight of the formulation.

14. The solid oral formulation of any one of claims 1 to 13, wherein content of the disintegrant other than the pregelatinized starch is 1 to 5% by weight with respect to 100% by weight of the formulation.

15. The solid oral formulation of any one of claims 3 to 14, wherein content of the pregelatinized starch is 15 to 30% by weight with respect to 100% by weight of the formulation.

16. The solid oral formulation of any one of claims 1 to 15, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof is 1 to 30% by weight with respect to 100% by weight of the formulation.

17. The solid oral formulation of any one of claims 1 to 16, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof is 5 to 20% by weight with respect to 100% by weight of the formulation.

18. The solid oral formulation of any one of claims 1 to 17, wherein content of the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof is 5 to 15% by weight with respect to 100% by weight of the formulation.

19. The solid oral formulation of any one of claims 1 to 18, further comprising an excipient.

20. The solid oral formulation of claim 19, wherein the excipient is a water soluble excipient.

21. The solid oral formulation of claim 20, wherein the water soluble excipient is mannitol.

22. The solid oral formulation of any one of claims 1 to 21, further comprising a lubricant.

23. The solid oral formulation of claim 22, wherein the lubricant is sodium stearyl fumarate.

24. The solid oral formulation of any one of claims 1 to 23, wherein the solid oral formulation is prepared by granulating a mixed powder comprising the (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof, the disintegrant, and the water soluble excipient by using a solution with the water soluble macromolecular binding agent dissolved therein.

25. The solid oral formulation of any one of claims 1 to 24, wherein a film coating is applied with a coating agent.

26. The solid oral formulation of claim 25, wherein the coating agent is selected from the group consisting of hypromellose, polyvinylpyrrolidone, and hydroxypropyl cellulose.

27. The solid oral formulation of claim 25 or 26, wherein the coating agent further comprises a plasticizer.

28. The solid oral formulation of claim 27, wherein the plasticizer is selected from the group consisting of polyethylene glycol, propylene glycol, triacetin, triethyl citrate, glycerin, glycerin fatty acid ester, and polyethylene glycol.

29. The solid oral formulation of any one of claims 25 to 28, wherein the coating agent further comprises a coloring agent.

30. The solid oral formulation of claim 29, wherein the coloring agent is titanium oxide and/or yellow iron sesquioxide.

31. The solid oral formulation of any one of claims 1 to 30 for treating and/or preventing a digestive system disease, a digestive system symptom, a neuropsychiatric disease, or a urinary system disease.

32. The solid oral formulation of claim 31, wherein the digestive system disease is an irritable bowel syndrome (IBS) with constipation or chronic constipation.

33. A composition for reducing or preventing the delay of drug dissolution, comprising a disintegrant.

34. The composition of claim 33, wherein the delay of drug dissolution is reduced or prevented after storage.

35. The composition of claim 33 or 34, wherein the delay of drug dissolution is reduced or prevented after an accelerated test (40° C./75% RH).

36. The composition of any one of claims 33 to 35, wherein the drug is (S)-4-amino-5-chloro-N-[{4-[(1-hydroxyacetyl-4-piperidinyl)methyl]-2-morpholinyl}methyl]-2-methoxybenzamide or a pharmaceutically acceptable salt thereof, or a hydrate or solvate thereof.

37. The composition of any one of claims 33 to 36, wherein the disintegrant comprises a cellulose-based disintegrant.

38. The composition of any one of claims 33 to 37, wherein the disintegrant comprises croscarmellose sodium or a combination of low substituted hydroxypropyl cellulose and pregelatinized starch.

39. The composition of any one of claims 33 to 38, wherein the disintegrant comprises croscarmellose sodium.

40. The composition of any one of claims 33 to 39, wherein the disintegrant comprises pregelatinized starch.

41. The composition of any one of claims 33 to 40, wherein the disintegrant comprises a combination of croscarmellose sodium and pregelatinized starch.

42. The composition of any one of claims 33 to 41, wherein the disintegrant comprises a combination of low substituted hydroxypropyl cellulose and pregelatinized starch.

43. The composition of any one of claims 33 to 42, which is used further in combination with a water soluble macromolecular binding agent.

44. The composition of claim 43, wherein the water soluble macromolecular binding agent is selected from the group consisting of polyvinylpyrrolidone, copolyvidone, hydroxypropyl cellulose, and polyvinyl alcohol.

45. The composition of any one of claims 38 to 44, wherein a portion of the pregelatinized starch that is soluble in cold water is 40% by weight or less.

46. The composition of any one of claims 38 to 44, wherein a portion of the pregelatinized starch that is soluble in water is 40% by weight or less.
